(19) United States
(12) Patent Application Publication
(10) Pub. No.: US 2007/0088211 A1
(43) Pub. Date: Apr. 19, 2007

(54) Method and apparatus for producing simulated fMRI data

(76) Inventors: Hu Cheng, Bloomington, IN (US); Qun Zhao, Gainesville, FL (US); William A. Edelstein, Schenectady, NY (US); G. Randy Duensing, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/452,605

(22) Filed: Jun. 13, 2006

Related U.S. Application Data
(63) Continuation-in-part of application No. 10/957,822, filed on Oct. 4, 2004.
(60) Provisional application No. 60/689,817, filed on Jun. 13, 2005.
(60) Provisional application No. 60/508,570, filed on Oct. 3, 2003.

(51) Int. Cl. A61B 5/05

(52) U.S. Cl. 600/410

(57) ABSTRACT

The subject invention relates to a method and apparatus for producing stimulated MRI data. In an embodiment, a remote-controlled “smart phantom” can produce simulated data. The simulated data can be acquired from a MRI system. The subject device can generate control signals and send the generated control signals to secondary coils/probes placed in the subject smart phantom. The control signals determine the current flow in the secondary coils/probes, which act as local spin magnetization amplifiers and thus produce regions of variable contrast to noise ratio. The control signals can be generated with various parameters, such as BOLD models, different levels of contrast-to-noise ratio (CNR), signal intensities, and physiological signals. Comparisons can be made with the widely-used simulated data by computers. Validation of the subject smart phantom can be performed with both theoretical analysis and data of human subjects.

### CROSS-REFERENCE TO RELATED APPLICATION

The present application claims the benefit of U.S. Provisional Application Ser. No. 60/689,817, filed Jun. 13, 2005; this application also claims priority to U.S. patent application Ser. No. 10/957,822, filed Oct. 4, 2004, which claims the benefit of U.S. Provisional Application Ser. No. 60/508,570, filed Oct. 3, 2003, all of which are hereby incorporated by reference herein in their entirety, including any figures, tables, or drawings.

The subject invention was made with government support under a research project supported by NIH grant no. RO1EB00974.

### BACKGROUND OF THE INVENTION

Over the course of the past decade, a variety of neuroimaging technologies allow the structure and function of the intact brain to be studied. This presents a tremendous opportunity to understand the human brain. Neuroimaging has the potential to reveal some of nature's most closely held and significant secrets, and informatics can assist in realizing this potential.

As defined in Neuroimaging Informatics Technology Initiative (NifTI) 2000 Workshop, “informatics, a computerized way to handle data, is used to design and implement the manner in which the imaging instruments capture signals generated by the brain, as well as the behavioral tasks used to probe particular brain systems, reconstruct the resulting signals, statistically analyze the data, and visualize the results”. By this definition, it is easy to find out that the informatics includes a variety of technologies, e.g., collection of data using new acquisition techniques (e.g. phase array coil), statistical analysis and processing of data, and visualization of data. However, the great potential of informatics in neuroimaging has been impeded by inadequate coordination regarding the development and distribution of the informatics tools needed to meet this challenge. Existing informatics tools have been developed separately, and now widely used software products are not adequately optimized for meeting the variety of needs of the broader community.

There exists a strong need that the conclusions drawn from fMRI studies regarding the relationship between functional activation signals and function of the brain should be based upon a clear understanding of the manner in which various tools affect the data at each stage of processing. Knowing that the operations performed by informatics tools are valid is important for interpreting the results of fMRI studies. In addition, comparing different tools to identify the conditions under which, and uses to which, they are best suited is significant information to both the researchers and clinicians. In this background section, some recent studies of fMRI activation simulation, statistical analysis tools, and parallel MRI reconstruction using phased array coils will be briefly reviewed.

Simulation of fMRI Activation and BOLD Signals

After conception and implementation of any new neuro-imaging informatics tools, validation is an important step to ensure that the procedure fulfills all requirements set forth at the initial design stage. Manufacturer's physical phantoms were first used for this purpose. However, these are typically just static phantoms filled with water or gel. The informatics tools must be evaluated using a comprehensive validation that requires additional use of simulated data since it is very difficult to establish ground truth with in vivo data. Experiments with simulated data permit controlled evaluation over a wide range of conditions, such as different Signal-to-Noise Ratio (SNR), contrast-to-noise ratio, and signal intensities. Such considerations have become increasingly important with the rapid growth of neuro-imaging.

Computer simulations of the Blood Oxygenation Level Dependent (BOLD) signals and neural activation have been developed by many researchers. In a recent publication (Sorenson, J. A. & Wang, X, ROC methods for evaluation of fMRI techniques. Magn. Reson. Med., 36:737-744 (1996)), the time course data was represented by a zero-baseline boxcar function. Gaussian noise was added to simulate physiologic “noise”, after which the data were convolved with a Poisson function to simulate hemodynamics smoothing and delay. Also, structured noise in the form of slopes to simulate signal drift and sinusoidal oscillations to simulate respiratory motion were added. These simulated fMRI responses were finally added to null data sets acquired on normal subjects, denoted as “human data”. In one study (Constable, R. T. et al. Quantifying and comparing region-of-interest activation patterns in functional brain MR imaging: methodology considerations, Magnetic Resonance Imaging, 16(3):289-300 (1998)), Gaussian-shaped activation signals were added with Gaussian distribution of white noise.

Another study (Morgan, V. L. et al. Comparison of functional MRI image realignment tools using a computer-generated phantom. Magn Reson Med. 2001 September; 46(3):510-4) discussed the development of a computer-generated phantom to compare the effects of image realignment programs on functional MRI (fMRI) pixel activation. The phantom is a whole-head MRI volume with added random noise, activation, and motion. It allows simulation of realistic head motions with controlled areas of activation. Without motion, the phantom shows the effects of realignment on motion-free data sets. Prior to realignment, the phantom illustrates some activation corruption due to motion. A recent paper (Desco, et al. Multiresolution analysis in fMRI: sensitivity and specificity in the detection of brain activation. Hum Brain Mapp. 2001 September; 14(1):16-27) presents a study that was undertaken to assess the performance of different wavelet decomposition schemes by making use of a “gold standard,” a computer-simulated phantom. As activation areas are then known “a priori,” assessments of sensitivity, specificity, ROC curve area, and spatial resolution can be obtained. This approach has allowed the study of the effect of different factors: the size of the activation area, activity level, signal-to-noise ratio (SNR), use of pre-smoothing, wavelet base function and order and resolution level depth. Recently, a Functional Data Simulator has been provided by SENSOR SYSTEMS (Medical Numerics, Inc., Sterling, Va.) in their software package MEDx. This functional data simulator (MEDx) is a graphical tool for constructing curves of signal intensity vs. time at a single pixel and then extrapolating to volumetric data. It can also be used to create synthetic fMRI data for similar exploration of the effects of noise and timing.

However, these studies, while providing a simulated activation signal, do not account for variations in the activation levels of different pixels and are unable to depict accurately the temporal response of the activation associated signal change. And the most important issue is that these simulations do not account for the real MR imaging environment (non-stationary rather than stationary, or dynamic rather than static), spin characteristics and the noises introduced during the whole imaging process, i.e. noises from MR power system, pre-amplifier, transmit/receive, and digitization (A/D) error.

By reviewing the literature, one can see that there are very limited measurements, such as the signal-to-noise ratio or contrast-to-noise ratio, where static phantom (a simple container full of liquid) has usually been used that provides static MR signals instead of dynamic signal changes. As for the fMRI research, systematic comparison of the analysis methods presented in fMRI cannot be conclusive if assessment is based only on the highly variable activity of the human brain. Calibrated, repeatable fMRI signals are needed for a reliable validation.

Statistical Analysis of Activation

In recent years, a variety of advanced high performance informatics tools have emerged for statistical pattern mapping of functional data and fMRI data visualization. Among them are the general-purpose fMRI analysis packages, such as AFNI, Brain Voyager™, BrainTools, FIASCO, fmristat, iBrain, Lyngby, MEDx, scanSTAT, and Statistical Parametric Mapping (SPM) as well as Special-purpose software packages, such as AIR, ANALYZE, ANIMAL, FreeSurfer, and SureFit. Qualitative characterizations and comparisons have been done (Gold et al. Functional MRI statistical software packages: a comparative analysis. Hum Brain Mapp. 1998; 6(2):73-84, Lange et al. Staatistical procedure for fMRI. Chapter 27 in Functional MRI, C. T. W. Moonen & P. A. Bandettini (Eds), Springer-Verlag (1999)). However, due to lack of a benchmark database, quantitative validation and comparisons of these tools are difficult and sometimes impossible.

The field of fMRI has been dominated by the notion of detectability of activation in a noisy background, and tremendous effort has been placed in the area of statistical signal detection methods. Among these statistical methods, some provide maps or images of descriptive statistics, such as z-score, statistical t-test, General Linear Model (GLM), and Kolmogorov-Smimov test. Generally speaking, all such maps represent a ratio of the activation magnitude to the measured signal variation.

Based on the differences in model, these techniques can be divided into two groups. The first is the parametric group. In general, parametric approaches depend on assumptions about the possible parametric families of distributions generating the fMRI data. They attempt to determine the value of a parameter or parameter vector that identifies a particular member among a large parametric family of plausible probability distributions that generate the fMRI data. The parametric group includes for example the statistical t-test, cross-correlation, and general linear model (Friston et al. Statistical parametric maps in functional imaging: A general linear approach. Human Brain Mapping, 2:189-210 (1995), Worsley & Friston Analysis of fRMI time-series revisited-again. NeuroImage, 2:173-181 (1995)), uni-variate time series models, analysis in frequency domain, and longitudinal data analysis.

The second group is the non-parametric approaches, which make no assumptions about the possible parametric families of distributions generating the fMRI data and are thus less dependent on a specific statistical model. While this seems attractive, there is another side. If parametric assumptions are roughly correct, then a parametric, model-based approach is superior to one that does not employ these assumptions. However, the difficulty is that it is usually difficult to know the properties of the true data-generating mechanism, and some statistical compromise between knowns and unknowns is often a pragmatic course of action. The non-parametric statistical procedures include, for example, the Kolmogorov-Smimov (KS) test, probabilistic analysis (Frank et al., Probabilistic analysis of fMRI data, Magnetic resonance in medicine, 39:132-148 (1998)), information-theoretic methods (Tsai et al., Analysis of functional MRI data using mutual information. Second International Conference of Medical Image Computing and Computer-assisted Intervention, 1679:473-480 (1999); Zhao, et al. 2002 Zhao, Q., Principe, J. C., Fitzsimmons, J., Bradley, M. M., Lang, P. J. (2001), “functional Magnetic Resonance Imaging Data Analysis with Information theoretic Approaches”, “Biocomputing”, edited by Panos Pardalos and Jose Principe, Chapter 9, pp. 159-174, Kluwer, 2002, and some modem computational non-parametric statistics (e.g., permutation, jackknife and bootstrap procedure).

However, there is very limited published information about how various tools affect the data at each stage of processing, what operations performed by informatics tools are valid, and under what conditions they are best utilized. A standard procedure or ground truth is needed for an objective characterization and comparison.

Establishing Ground Truth

Informatics tools are important both to performing neuro-imaging studies, and to understanding the results. After conception and implementation of fMRI informatics tools, validation is an important step to ensure that the procedure fulfills all requirements set forth at the initial design stage. Though these tools must be evaluated on real data, a comprehensive validation should ideally involve the additional use of simulated data with known parameters, since it is very difficult to establish ground truth with in vivo data. Qualitative analyses of various informatics tools have been done using computer-simulated functional activation signals. However, these computer-simulated data are usually simply overlapped on top of anatomic images, they cannot reflect the real MR imaging process, such as the MR spin characteristics and noises introduced during the process, i.e. noise from MR power system, pre-amplifier, transmit/receive, and digitization (A/D) error. Besides, due to lack of a highly characterized data set available to the research community, it is difficult to make an objective comparison of the fMRI informatics tools. A quantitative measurement tool is needed to provide data that presents the MR imaging process, the ground truth, in order to characterize and compare informatics tools.

Defining the Effect of Statistical Analysis Tools

Informatics tools are key to all stages of fMRI. However, there is very limited published information about several fundamental aspects of informatics tools. These issues include a clear understanding of the manner in which various tools affect the data at each stage of processing, the knowledge of under what conditions operations performed by informatics tools are valid, and comparisons of different tools to identify the conditions under which they are best utilized.

Calibration of different fMRI Experiments

Currently, numerous fMRI exams are conducted on various MRI scanners (e.g., General Electric Medical Systems, Siemens Medical Solutions, Philips Medical Systems) at different field strength (e.g., 1.5 T and 3.0 T) and with different pulse sequences (e.g., EPI, Spiral), head coils (e.g., birdcage, phased array) or protocols. With so many variables, comparison of the results become extremely difficult, making it uncomfortable to share the findings from different research groups. Therefore, a calibration tool which acts as a reference or benchmark of the specific exam is highly needed to provide useful information for comparison of the results.

Accordingly, considering all these problems, there is a need for a physical fMRI simulation device or phantom. There is a need for a device which can provide variable contrast of signals, with intensity changes following the BOLD signals, and can allow the optimization of imaging protocols. Also, there is a need for a device that can produce signals, such that the acquired signals from such a device can provide a gold standard for characterization, validation and comparison of various informatics tools. Furthermore, there is a need for a physical phantom which can provide means for instantly checking system performance. In addition, there is a need for a device or phantom that can provide one or more of the following features: a complete MR system and MR platform independent; have complete control over timing or delays, signal intensity level, motion, and physiological “noise” such as cardiac and respiratory signals; provide self-testing for quality control; and be easy to use and transportable.

### BRIEF SUMMARY OF THE INVENTION

The subject invention relates to a method and apparatus for producing simulated fMRI data. In an embodiment, the subject invention pertains to a remote-controlled “smart phantom” that can produce simulated fMRI data. In an embodiment, the simulated fMRI data can first be acquired from a MRI system. The subject device can generate control signals and send the generated control signals to secondary coils/probes placed in the subject smart phantom. The control signals can determine the current flow in the secondary coils/probes by adjusting the impedance of the secondary coils/probes, which can act as local spin magnetization amplifiers and thus produce regions of variable contrast to noise ratio. The control signals can be generated with various parameters, such as hemodynamic response models and different levels of enhancement and physiological noises. Comparisons can be made with the widely-used simulated data by computers. Validation of the subject smart phantom can be achieved with theoretical analysis and/or with data of human subjects.

The data associated with the use of the subject smart phantom can be used to establish a benchmark database, an Informatics Development and Support Database for fMRI. This database can also be used to document the performance of registered informatics tools for storage with respect to the user-adjustable informatics parameters, the user-selected data sets processed, and the resulting visualization.

The subject smart phantom can enable quantitative and objective characterization and comparison. For example, functional activation detection can be investigated and compared among the general-purpose and/or other statistical analysis packages. Sensitivity and specificity can be validated and compared by using the Receiver Operation Characteristics (ROC) curve under different levels of signal intensities, CNR, spatial resolution, and physiological spectral background. Various methods of motion correction can be characterized and compared using data acquired from accurately controlled motion of the subject smart phantom. Spatial and temporal filtering can also be characterized and compared.

### DETAILED DESCRIPTION OF THE FIGURES

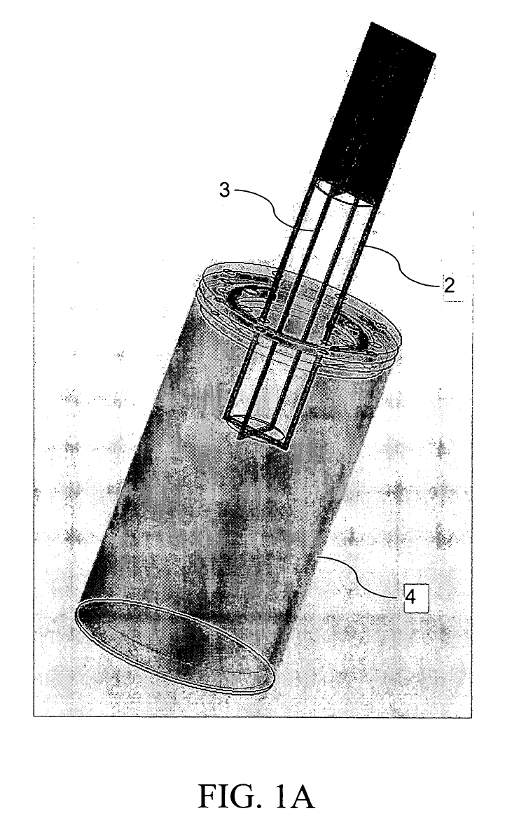

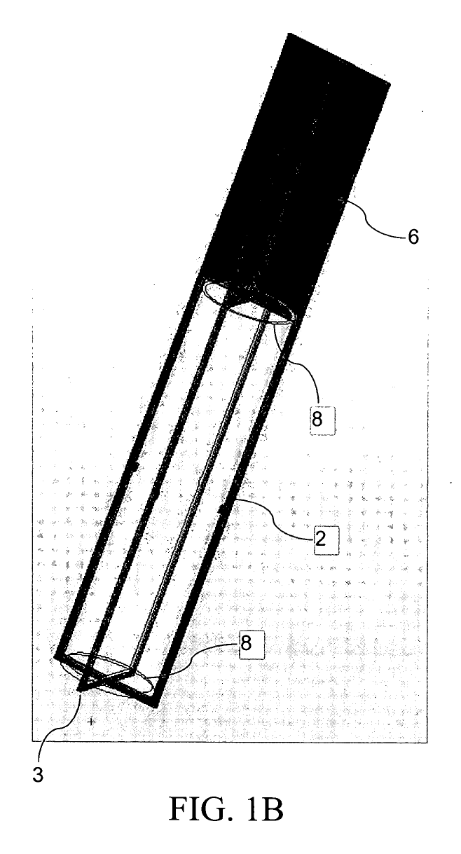

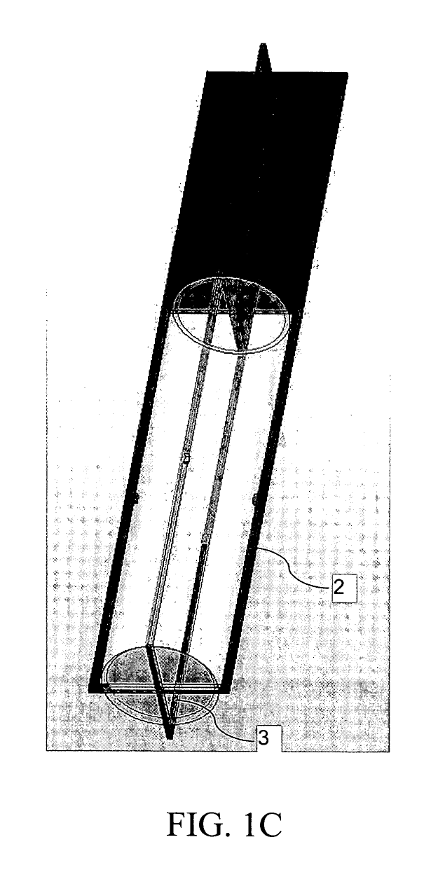

FIGS. 1A-1C show a specific embodiment of a phantom in accordance with the subject invention incorporating two planar rectangular loops.

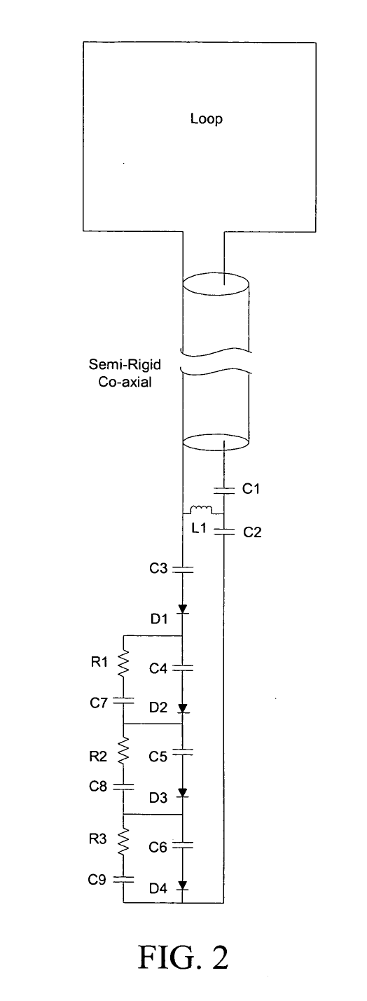

FIG. 2 shows a schematic diagram of a loop of a quadrature secondary coil incorporated with the phantom shown in FIGS. 1A-1C.

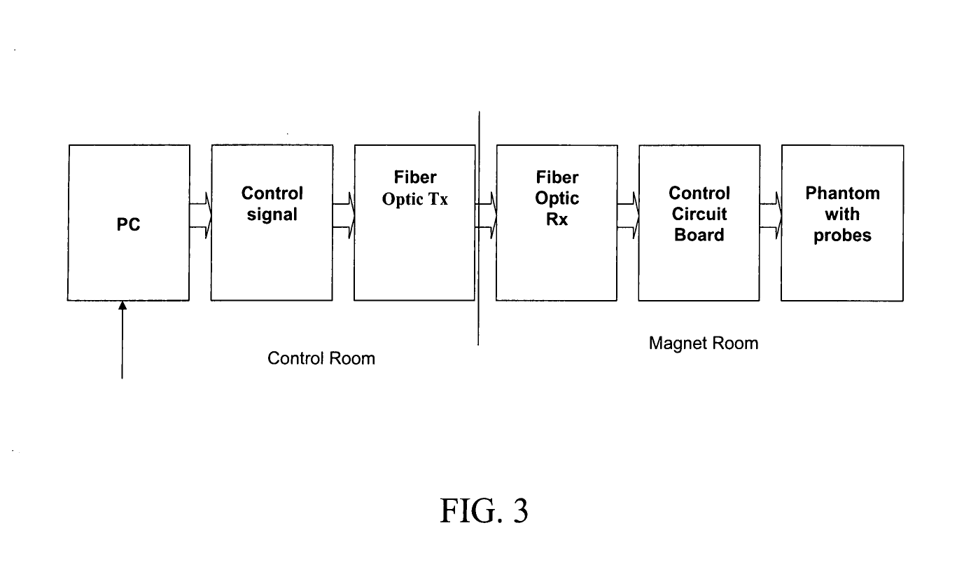

FIG. 3 shows a diagram of a specific embodiment of a smart phantom implementation system in accordance with the subject invention.

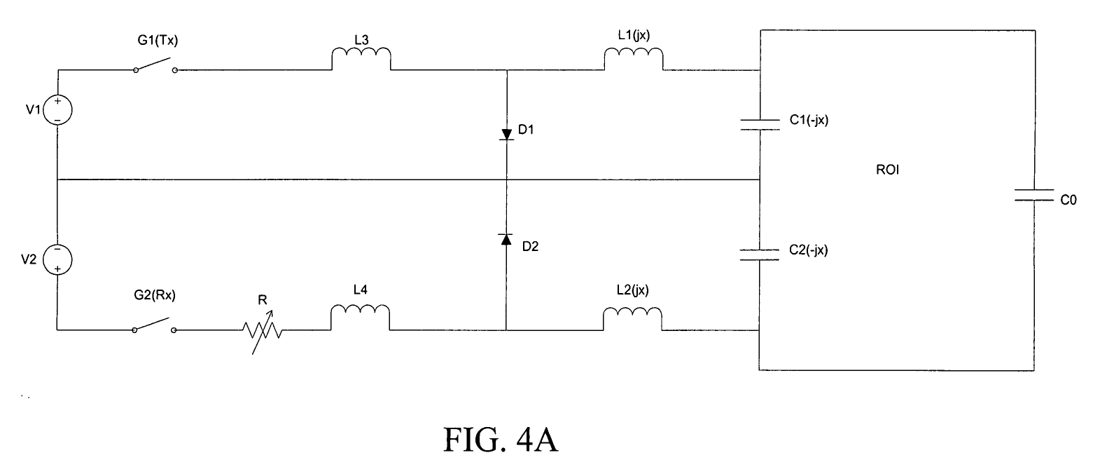

FIG. 4A shows a circuit schematic in accordance with a specific embodiment of a coil and circuitry for incorporation with a phantom.

FIG. 4B shows a circuit schematic in accordance with a specific embodiment of a coil and circuitry for incorporation with a phantom.

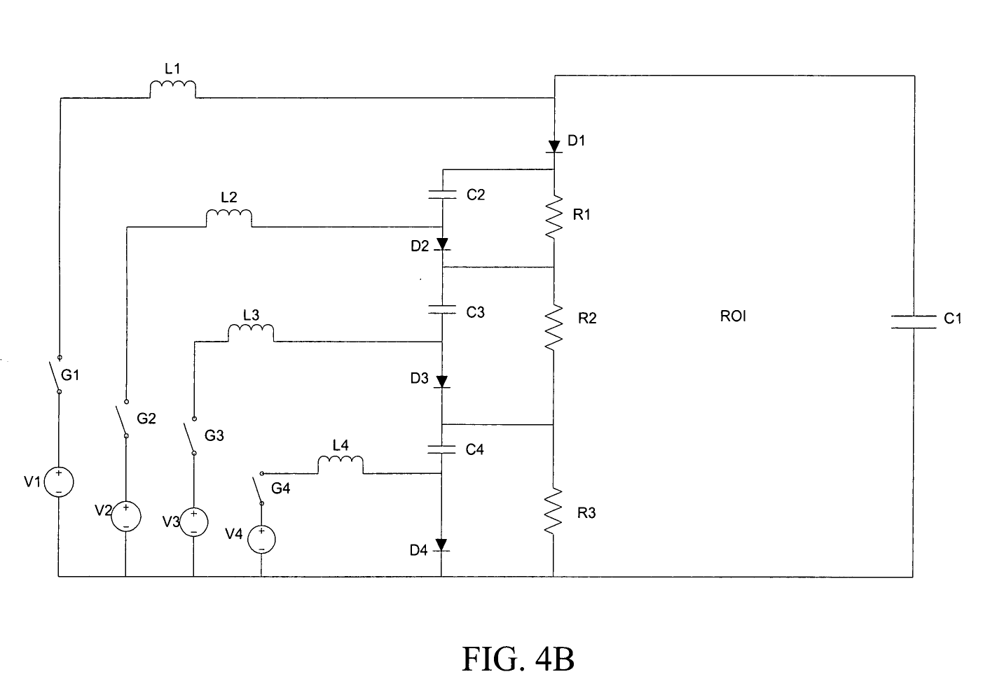

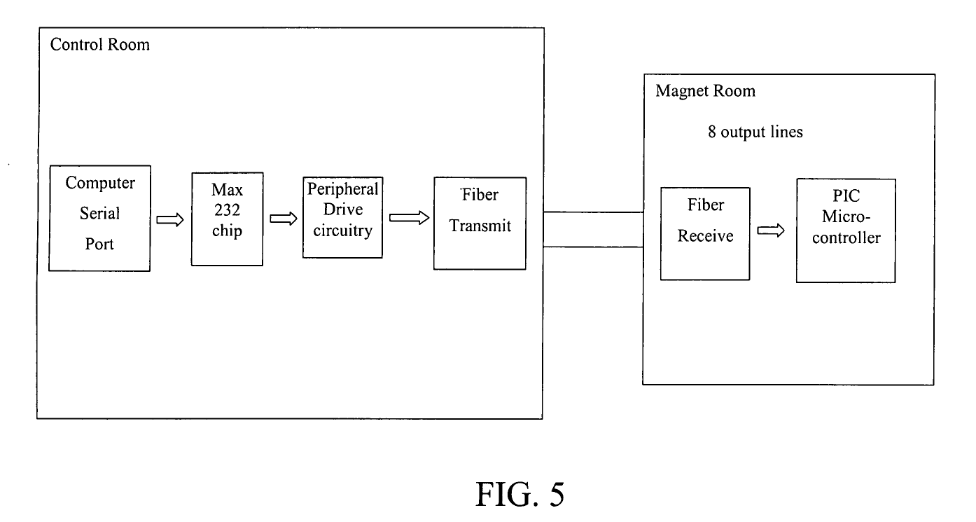

FIG. 5 shows an embodiment of an implementation incorporating the subject phantom in accordance with the subject invention.

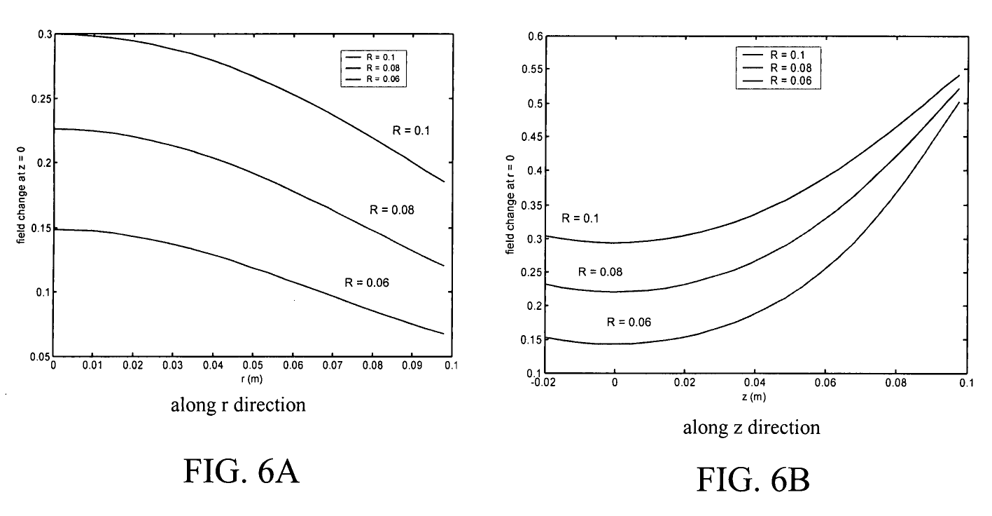

FIGS. 6A and 6B show field distortions in the radial and longitudinal directions, respectively, for a simulation of a cylindrical phantom of fixed length in the z direction.

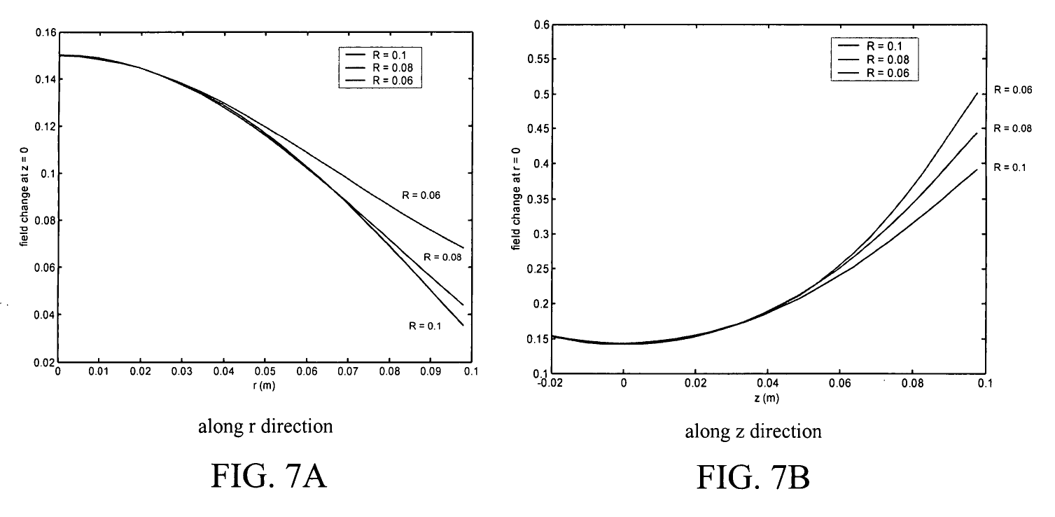

FIGS. 7A and 7B show a comparison of the field distortions for three radius by moving the curves in FIGS. 6A and 6B, respectively, to coincide at r=0 or z=0.

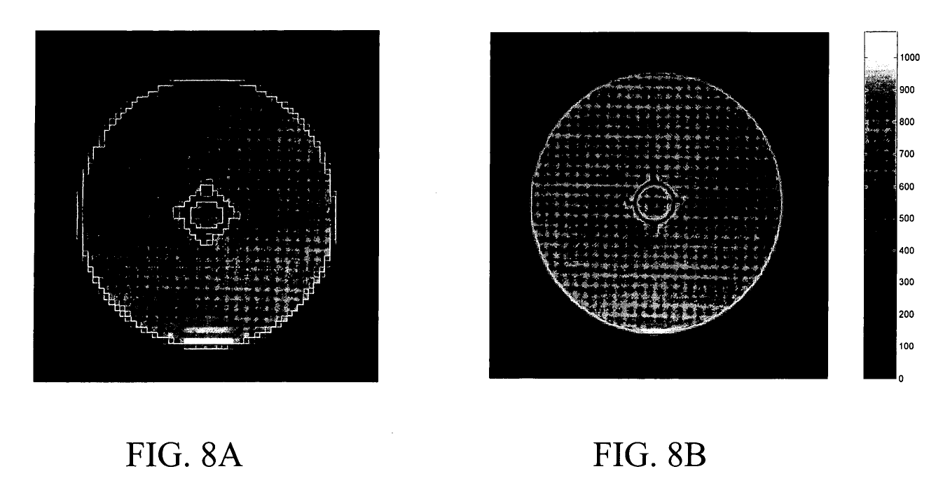

FIGS. 8A and 8B show an EPI (SLT=5 mm, TE=54, FOV=160 mm) image and a Spin-echo image (same slice, TR/TE=552/16) of a specific embodiment of the subject smart phantom acquired in a Siemens Symphony system.

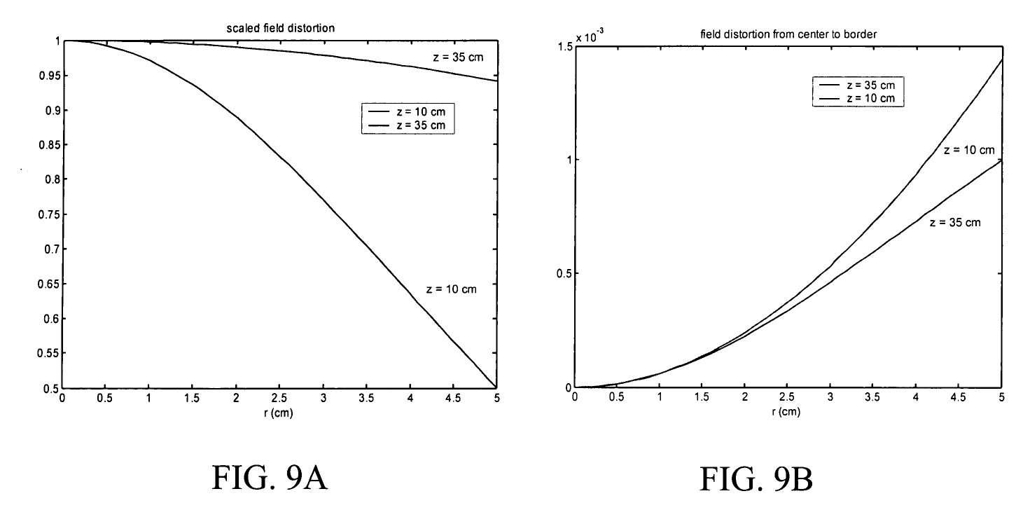

FIGS. 9A and 9B show the field distortion for z=10 cm and z=35 cm, normalized at r=0 and the field distortion for z=10 cm and z=35 cm, scaled to make the overall change from r=0 to r=5 close to each other, respectively.

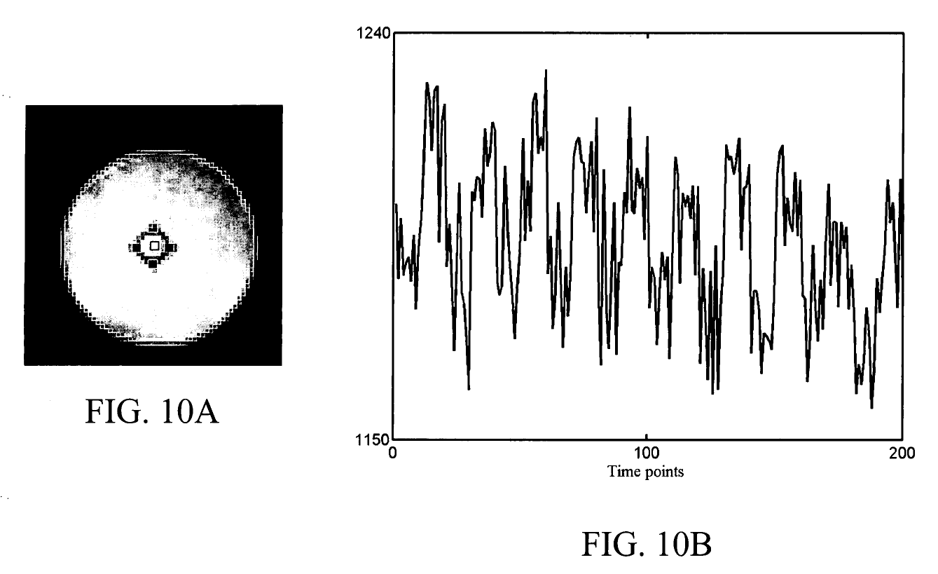

FIGS. 10A and 10B show an EPI image of an axial slice of an embodiment of a phantom in accordance with the subject invention and the BOLD-similar time course of the mean signal in the ROI with SNR=73, respectively.

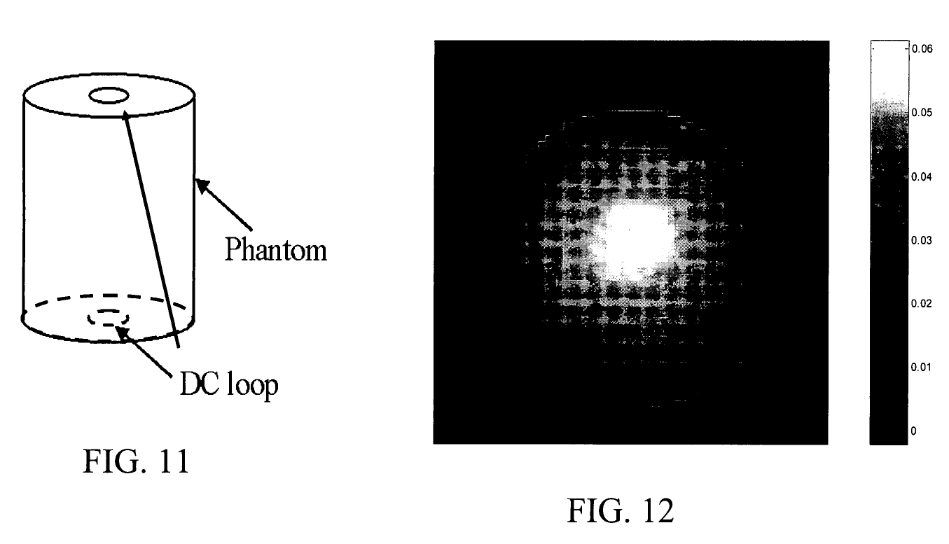

FIG. 11 shows an embodiment of the subject phantom having a cylindrical shape and having a loop attached to each end of the phantom such that the axis of the cylinder intersects the centers of the two loops.

FIG. 12 shows a difference image from an ‘on’ state and and ‘off’ state for a specific embodiment of the subject invention.

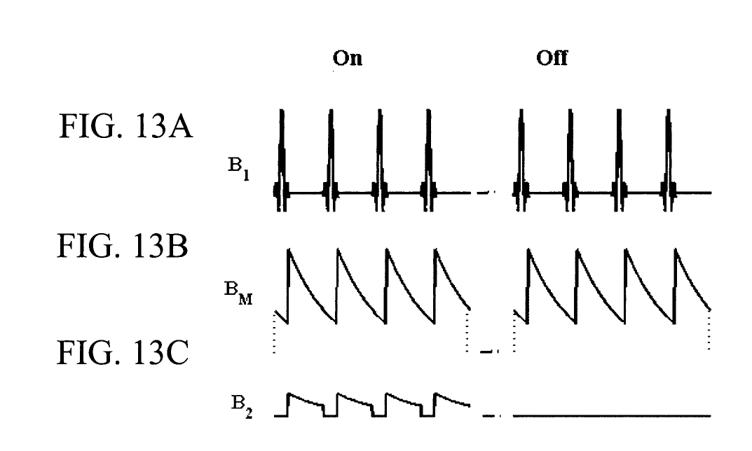

FIG. 13A shows the form a pulse pattern of an excitation RF magnetic field B₁, that can be used to excite a sample material in accordance with an embodiment of the subject invention.

FIG. 13B shows the form of the rotating magnetization M of a sample material located in a static magnetic field B₀ and excited with the excitation RF magnetic field B₁ shown in FIG. 13A.

FIG. 13C shows the form of the induced RF magnetic field B₂ in a RF coil position proximate to the sample material for which the rotating magnetization is shown in FIG. 13B, wherein the RF coil is decoupled during the last portion of the plot.

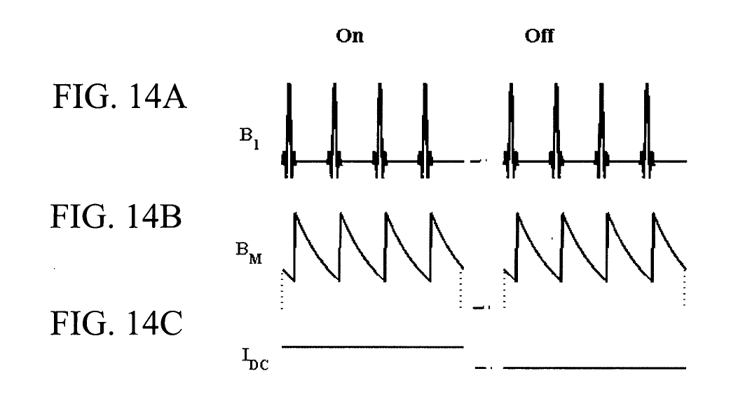

FIG. 14A shows the form a pulse pattern of an excitation RF magnetic field B, that can be used to excite a sample material in accordance with an embodiment of the subject invention.

FIG. 14B shows the form of the rotating magnetization M of a sample material located in a static magnetic field Bₒ and excited with the excitation RF magnetic field B₁ shown in FIG. 14A.

FIG. 14C shows the DC current that can drive one or more DC coils associated with a magnetic field parallel to the static magnetic field Bₒ to produce a static magnetic field opposed to field B,, or additive with field Bₒ, in accordance with an embodiment of the subject invention.

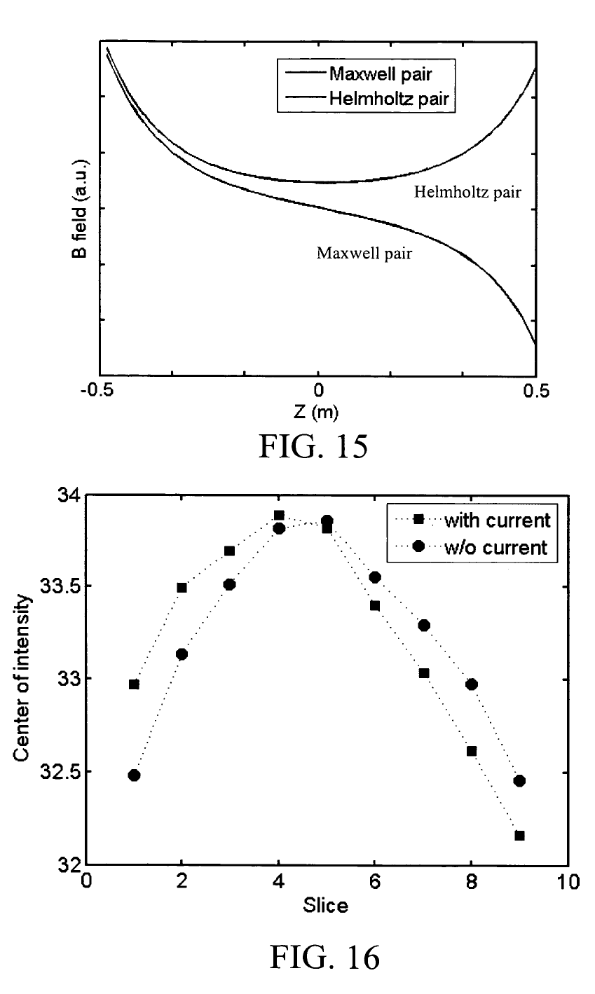

FIG. 15 shows a plot of the change in the B field strength parallel to the Bₒ field for two coils placed symmetrically with respect to Z=0 where Bₒ is parallel to the Z axis and driven as a Maxwell pair configuration or a Helmholtz pair configuration, in accordance with an embodiment of the invention.

FIG. 16 shows the center of intensity for 9 slices of a spherical phantom, with and without current in the two loops, in accordance with an embodiment of the invention.

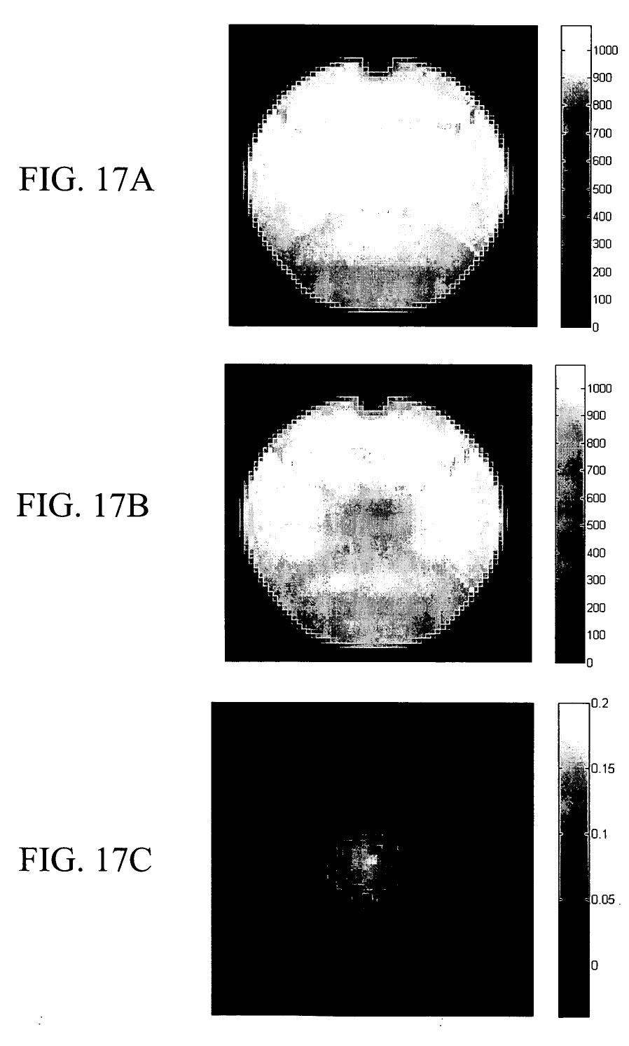

FIG. 17A and 17B show an image of the middle slice of a spherical phantom without current, and with current, respectively.

FIG. 17C shows a difference image between the image of FIG. 17B and the image of FIG. 17A, divided by the image of FIG. 17A.

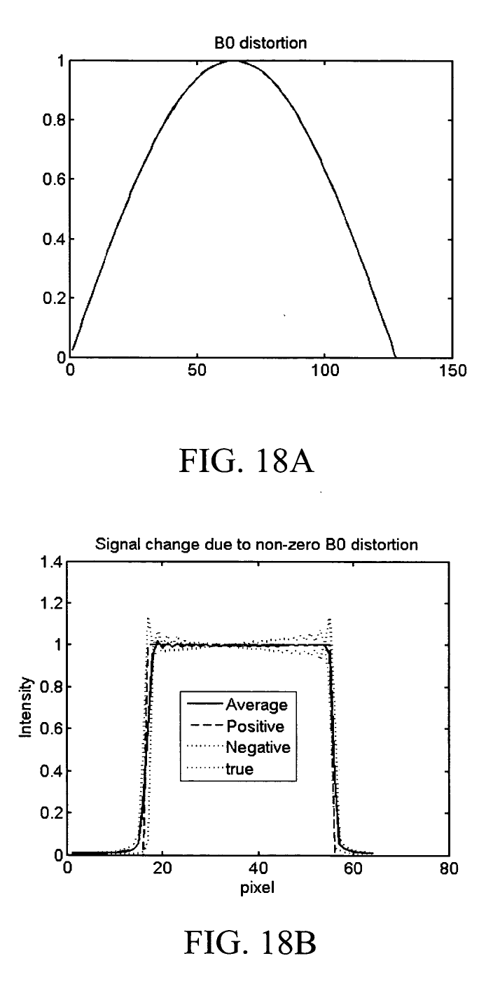

FIG. 18A and 18B show the profile of the distortion in Bₒ field, and a comparison of different signal changes due to the distortion of the Bₒ field shown in FIG. 18A, respectively.

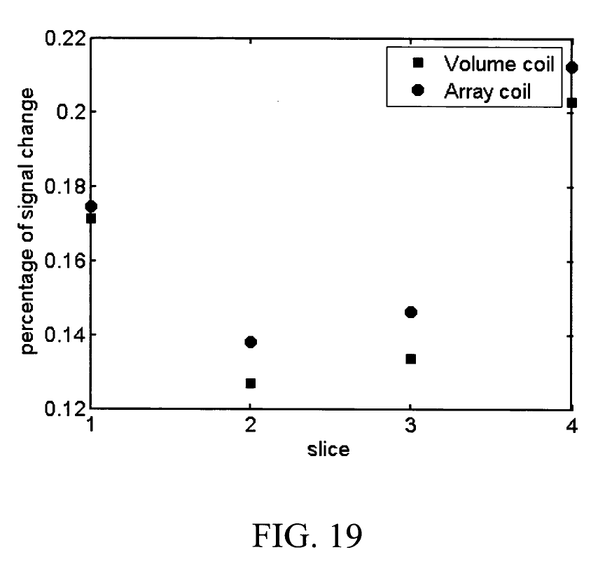

FIG. 19 shows a plot of contrasts of an embodiment of a phantom in accordance with the subject invention for a volume coil and for an array coil.

### DETAILED DISCLOSURE OF THE INVENTION

The subject invention relates to a method and apparatus for producing simulated fMRI data. In an embodiment, the subject invention pertains to a remote-controlled “smart phantom” that can produce simulated fMRI data. In an embodiment, the simulated fMRI data can first be acquired from a MRI system. In this sense, the MRI data is data actually acquired from a MR scanner, but the MRI data is referred to as “simulated” fMRI data because it is not acquired from the dynamic activity of a patient. The subject device can generate control signals and send the generated control signals to secondary coils/probes placed in the subject smart phantom. The control signals can be used to adjust the impedance of the secondary coils/probes so as to determine the current flow in the secondary coils/probes, which can act as local spin magnetization amplifiers and thus produce regions of variable contrast to noise ratio.

The subject invention can involve the placement of a sample material in a region of interest in a static magnetic field Bₒ. The sample material in the static magnetic field Bₒ will have a magnetization M. One or more RF coils can be positioned in the static magnetic field proximate the sample material. For purposes of ease of description, the following is a discussion of a single RF coil. The RF coil is associated with a magnetic field having a component perpendicular to the static field Bₒ in the region of interest. Of course, the magnetic field associated with the RF coil changes direction as a function of position and, as the static magnetic field Bₒ has a fairly uniform direction in the region of interest, the relative direction of the magnetic field associated with the RF coil and the static field Bₒ will change as a function of position as well. Preferably, the magnetic field associated with the RF coil is perpendicular to the static filed Bₒ in the region of interest More preferably, the RF coil is planar and has a normal perpendicular to the static field Bₒ. This discussion can be applied to additional RF coils employed in multi-RF coil embodiments.

An excitation RF magnetic field B₁ is applied to the sample material so as to tilt the magnetization M of the sample material. Referring to FIG. 13A, the excitation field B₁ can be applied as a series of pulses to form a pulse pattern. The RF coil can be decoupled during each pulse to reduce or eliminate fields that would be produced by the RF coil by the field B₁. After each pulse of field B₁, the RF field produced by the rotating magnetization M induces a voltage V in the RF coil. The induced voltage produces a current

$$I = \frac{V}{Z}$$

in the RF coil, where Z is the impedance of the RF coil. The current I flowing in the RF coil then produces an induced RF magnetic field B₂. FIG. 13C shows the form of a B₂ field that can be produced in an embodiment of the invention. The B₂ field shown in FIG. 13C is positive for the early pulses because the impedance of the RF coil is adjusted accordingly. The B₂ field shown in FIG. 13C is zero for the later pulses because the RF coil is decoupled. Adjustment of the impedance Z of the RF coil alters the induced magnetic filed B₂ for the same static magnetic field Bₒ, the same sample material, and the same excitation RF magnetic field B₁. Adjustment of the impedance of the RF coil can be accomplished by adjusting the resistance of the RF coil.

The means for detecting the net RF field over the region of interest can then measure the net RF field Bₙₑₜ. This means for detecting Bₙₑₜ can be, for example, one or more receive coils. A voltage is produced in each receive coil due to Bₙₑₜ to produce a portion of a MRI signal. When the RF coil is not decoupled, Bₙₑₜ is the vector sum of B_{M} and B₂, after exciting the sample material with excitation field B₁, where B_{M}=μₒM, M is the rotating magnetization of the sample and μₒ is the magnetic permeability of free space. When the RF coil is decoupled, Bₙₑₜ is Bₘ after exciting the sample material. In an embodiment, excitation field B₁ be delivered in pulses separated by periods where B₁ is turned off, such that the net RF field can be measured between adjacent pulses of excitation field B₁. The means for detecting can then provide a first portion of an MRI signal when B₂ is non-zero and a second portion of the MRI signal when the RF coil is decoupled (or has very high impedance) and B₂ is zero. These two portions of the MRI signal can then be used to produce simulated fMRI data. In a specific embodiment, the second portion of the MRI signal can result from detection of Bₙₑₜ when B₂ is non-zero and the impedance of the RF coil has been adjusted with respect to the detection of Bₙₑₜ to produce the first portion of the MRI signal, in contrast to decoupling the RF coil during the production of the second portion of the MRI signal.

The resulting simulated fMRI data can be compared with the known excitation pattern to analyze the simulated fMRI data. In specific embodiments, the simulated fMRI data includes a portion of a MRI signal when the RF coil is decoupled, representing no stimulation of the simulated patient and a portion of the MRI signal when the RF coil is not decoupled representing stimulation of the simulated patient. As in actual fMRI measurements, various permutations of stimulation and no stimulation can be utilized. For example, the stimulation can be pulsed.

In a specific embodiment, the RF coil is positioned parallel to the static magnetic field Bₒ such that the RF coil is associated with an RF magnetic field that is perpendicular to the static magnetic field. Stated another way, for an embodiment using a planar coil, the normal of the plane of the coil is perpendicular to the static magnetic field. Although it is preferred to used RF coils associated with RF magnetic fields, perpendicular to the static field Bₒ, RF coils positioned at an angle to the static magnetic field Bₒ can also be utilized. Of course, with most RF coils the direction of the associated magnetic field changes as a function of position, such that the relative direction of the static field Bₒ and the associated magnetic field changes with position as well. In this way, the relative directions of the associated field and the static field Bₒ may be perpendicular over a certain region. In an embodiment, this can be considered the region of interest.

One or more additional RF coils can also be positioned proximate the sample material in much the same way as the first RF coil. Preferably, an additional RF coil is at an angle with respect to first RF coil, such that the additional RF coil is associated with an RF magnetic field that has a component perpendicular to the static magnetic field and perpendicular to the component of the first RF coil that is perpendicular to the static magnetic field. For example, if the static magnetic field is parallel to the Z-axis and the first RF coil is associated with an RF magnetic field having a component in the X direction, the additional RF coil preferably is associated with an RF magnetic field having a component in the Y direction. If the first RF coil and the additional RF coil are planar, another description is that the normal to the plane of the first RF coil has a component in the X direction and the additional RF coil has a component in the Y direction. A most preferred embodiment involves two planar RF coils that are both parallel to the static magnetic field and the normals to the planes of the two RF coils are perpendicular to each other. Although planar coils may be preferred in some applications, a variety of RF coils can be utilized, including, but not limited to the following: Aldermann-Grant, Helmholtz, ring, square, rectangular, and birdcage.

The subject invention also pertains to the incorporation of one or more coils for altering the net static magnetic field parallel to the static magnetic field Bₒ, by, for example, producing dc magnetic fields parallel to the static magnetic field Bₒ. Such dc field coils can be used in conjunction with one or more RF coils as discussed above or can be used without the RF coil(s). The term dc coil can be used to refer to coils used to alter the net static magnetic field parallel to the Bₒ field as the current driving the coils can be switched on and off, as desired, and can vary in magnitude and direction, as desired, as well. In an embodiment, such dc coils can be driven with an alternating current that has an average value of zero. The coils can produce strong enough magnetic fields perpendicular to the static magnetic field Bₒ so as to alter the static magnetic field parallel to the static magnetic field B,, and/or can produce magnetic fields parallel to the static magnetic field Bₒ. Preferably the dc field coil is associated with a dc field parallel to Bₒ in the region of interest. Most preferably, the dc field coil is a planar coil having a normal parallel to Bₒ. The dc field coil(s) then change the net static field experienced by the sample material located in the region of interest. Although it is described as changing the static field, it is understood that the dc current driving the dc coil can be made to vary such that the static field in the direction of Bₒ is in fact a dynamic field. In fact, the dc coil(s) can be driven with dynamically varying currents that include a model of one or more physiological functions, or other conditions to be modeled, such as patient's breathing. Again, the strength and direction of the magnetic fields associated with the dc coil(s) varies with position so that the dc coil(s) changes the field in the direction of the static field Bₒ as a function of position. In an embodiment, the current used to drive the one or more coils to alter the static magnetic field in the Bₒ field direction has at least two different values, so that the net static magnetic field in the Bₒ field direction changes.

For example, if the static field coil produces a static field component in the direction parallel to Bₒ of Bₛ, then the net static field in the direction parallel to Bₒ is Bₒ+Bₛ, if B_{S} is in the same direction as Bₒ, and Bₒ−B_{S}, if B_{S} is in the opposite direction of Bₒ. This change in the static field can alter the resulting magnetic field B_{M} of the rotating magnetization M. FIGS. 14A-C show an excitation field B₁, the resulting magnetic field B_{M} of the rotating magnetization M, and the DC current that can drive a coil for producing a static magnetic field parallel to the direction of Bₒ, respectively, in accordance with an embodiment of the subject invention. In a further embodiment, the excitation of FIG. 14A and the DC current of FIG. 14C can be applied to the embodiment of FIG. 11 having only one DC loop.

In an embodiment, the current that drives the one or more coils for altering the static magnetic field in the Bₒ field direction changes the MRI signal intensity less than an amount that would cause the loss of spatial information, for a given MR scanner gradient field. In a specific embodiment, the MRI signal intensity is changed less than 5%. In another embodiment, the MRI signal intensity is changed less than 4%.

A variety of sample materials can be utilized in accordance with the subject invention. The sample material can be chosen to have magnetic properties of another object or material such as a human brain, a human heart, and/or a human bone. In a specific embodiment, a gel, such as an agarose gel, can be used. The gel can incorporate substances which alter its properties. An example includes an agarose gel with copper sulfate. In this example, the concentrations of agarose and copper sulfate can be adjusted to match a particular material such as human brain tissue. In a specific embodiment, the sample material can be selected so as to have a T1 value, T2 value, and/or T1/T2 ratio in a certain range. In a specific embodiment, the sample material can be a human head or other portions of a patient or object to be imaged.

All or a portion of the sample material can also be moved during the production of the simulated fMRI data. Such motion can be periodic, if desired, so as to model, for example, breathing. In additional embodiments, the sample material can flow through a conduit during the production of the simulated fMRI data. Such flowing of the sample material can be used to, for example, model blood flow. The properties of the flowing sample material can be adjusted during the production of the fMRI data. This can be utilized to model a dynamically changing model material. One or more of the RF coils and/or static field coils can be moved during production of the simulated fMRI data. The shape of the sample material can also be changed to model a certain object and/or material. For example, a cavity within the sample material can be expanded and contacted during the production of stimulated fMRI data and/or the sample material can be expanded and/or contracted. In an embodiment, one or more RF shields can be incorporated to shield some of the sample material from the magnetic field B₂ and/or from the excitation field B₁, in order to assist in the creation of a known enhancement pattern with known characteristics.

Preferably, the sample material has a high proton density and a T2 relaxation that is long enough to provide sufficient stimulated fMRI data, where the T2 relaxation time characterizes the decay of the transverse component of the magnetization caused by spin-spin interaction after excitation. A gel having water can be used and can have additives to adjust the T2 and T1 of the gel in order to have the desired properties. In an agarose gel, copper sulfate can be used to adjust the T1. Human brain grey matter has a T1 and T2 of approximately 1000 ms and 90 ms, respectively. The sample material can also be water doped with different ions, oils, and/or organic material such as polyvinyl alcohol so as to have T1 and/or T2 comparable to a desired tissue to model, such as a human brain.

In a specific embodiment, the subject invention pertains to a functional magnetic resonance imaging (fMRI) activation simulator and a method of providing simulated fMRI activities. The subject fMRI activation simulator can generate simulated functional changes based on known, controllable signal input. When placed in an MRI scanner the subject phantom can produce calibrated and repeatable fMRI activities, so as to permit an objective, systematic comparison of fMRI analysis methods. The controllable signal input can allow such factors as hemodynamic delay to be taken into account, and can allow control of the shape of the output signal so as to, for example, control contrast.

The subject phantom can simulate various hemo-dynamic response models, smoothing and delay, and cardiac and respiratory “noise”. The subject phantom can simulate the neural activation signal and produce a signal intensity change resembling the physiological data. The subject phantom can provide a means to characterize, validate, and compare informatics tools, as well as be used for MRI quality control.

FIGS. 1A-1C shows an embodiment of the subject smart phantom incorporating two planar rectangular RF probes/coils 2, 3 placed into a cylinder 4. FIG. 1A shows the probes 2, 3 during insertion into cylinder 4. Excitation of the RF coils induces magnetic field changes and thus produce regions of variable MR image contrast to noise. The excitation of the RF coils can be under the control of a computer. The RF coils can be oriented such that the magnetic field associated with driving a current through the RF coil has a component perpendicular to the main static magnetic field of the MRI MR scanner into which the subject phantom is placed. Stated another way, the normal to the plane of the planar RF coils has a component perpendicular to the main static magnetic field Bₒ. In the embodiment shown in FIGS. 1A-1C, the RF probe is a quadrature secondary coil, having two planar rectangular loops in parallel with the static magnetic field, such that the normal of each of the planes associated with the planar loops is perpendicular to the main static magnetic field Bₒ. In the embodiment of FIGS. 1A-1C, the two planar loops are placed at a 90 degree angle with respect to each other. In alternative embodiments, the two loops can be placed at other angles with respect to each other or can be parallel to each other. In the embodiment shown in FIGS. 1A-1C, the two loops are planar rectangular and are positioned to form an inner cylinder 8, where the inner cylinder 8 shown in FIGS. 1A-1C in outline form. Sample material can be placed inside the inner cylinder 8 of the subject phantom in sufficient proximity to the RF coils that the rotating magnetization of the sample material can reach the RF coils so as to have a meaningful impact on the MRI measurements. In a specific embodiment, a container having a sample material can be located such that at least a portion of the sample material is located within the inner cylinder 8 formed by the surface loops. In an embodiment, the sample material can be located within a cylindrical container and the cylindrical container can be placed inside the two loops.

Referring to the embodiment shown in FIGS. 1A-1C, the two planar rectangular loops travel up the outside of the structure from the bottom and the travel toward the central axis of the inner cylinder 8 at the top of the inner cylinder formed by the loops, leaving the top portion 6 to house the circuitry of the loops, which can be used to, for example, control the impedance of the loops.

A schematic diagram of a specific embodiment of a loop in accordance with the subject invention is shown in FIG. 2. In this embodiment, the diode symbol represents a PIN diode. During transmit, or excitation of the sample material with excitation RF magnetic field B₁ by the MR Scanner, the PIN diodes D₁, D₂, D₃, and D₄ can be reverse biased such that no current flows in the circuit, such that the RF coil is decoupled. During receive, or collection of data by the MR Scanner, the resistors of the resistor array can be switched in and out, by turning on and off each individual PIN diode. The resistor array can be controlled so as to adjust the impedance of the RF coil in order to achieve a desired degree of signal enhancement. When a sample material is placed in the static magnetic field of the MR Scanner and excited with an excitation RF magnetic field B₁, the magnetization of the sample material M will rotate. The rotating magnetization M induces a voltage in the RF coil, which induces a current to flow in the RF coil. The current flowing in the RF coil produces an induced magnetic field B₂. In an embodiment, the loop can be tuned such that it is purely resistive at resonance. In an embodiment, the induced current can be 90 degrees out of phase with the applied magnetization, such that the signal from the loop and the signal from the sample material magnetization can be added in quadrature. The control signal for controlling the impedance of the RF coil can be produced by computer simulation, which can provide, in various embodiments, one or more of the following functions:
(1) A range of enhancement level (for example from 1% to 10%) can be provided over regions of interest. Positive and negative enhancement levels can be provided and, in an embodiment, enhancement levels can between about 0.25% and about 10% can be provided. Enhancement levels higher than 10% can also be achieved.
(2) Typical hemo-dynamic waveforms (e.g., Boxcar, Gamma, Gaussian) can be provided.
(3) Duty cycle (on-off duration) control can be provided, such that each cycle can have a variable or fixed stimulus/on period and a variable or fixed control/off period.

A synchronization signal (e.g., transmit unblank from MR scanner) can be utilized to start the computer sending the control signal, in synchronization with the functional scan.

A diagram of a specific embodiment of a smart phantom implementation system in accordance with the subject invention is shown in FIG. 3. A personal computer (PC) can be used as a main control unit, which sends out the control signals. In a specific embodiment, the signal transmission between the PC (in the control room) and the subject phantom (in the magnet room) can be done using fiber optics. A shielded control circuit board can be used to magnify the signal before it is sent to the coils/probes in the subject phantom.

The subject invention can also be utilized to evaluate an algorithm for analyzing fMRI data. In an embodiment, simulated fMRI data with a known enhancement pattern can be provided, the simulated fMRI data can be analyzed via an algorithm for analyzing fMRI data to create a prediction of the known enhancement pattern and the prediction of the known enhancement pattern can be compared to the known enhancement pattern. In a further embodiment, one or more parameters of the algorithm can be adjusted and the simulated fMRI data can be reanalyzed. The adjusted prediction of the known enhancement pattern can be compared to the known enhancement pattern. The adjustment of parameters and comparisons can be repeated until the comparison indicates an effective match between the adjusted prediction and the known enhancement pattern is achieved. If desired, the adjusted algorithm can then be utilized to analyze fMRI data from a patient. In a specific embodiment, the method of evaluating an algorithm for analyzing fMRI data can utilize simulated fMRI data produced via the use of the subject phantom as described herein.

There are many popular software packages that utilized methods for analyzing fMRI data. We have compared SPM (statistical parametric mapping), FSL (FMRIB Software Library), and BrainVoyager™. Another example of a software package that analyzes fMRI data is AFNI (Analyses of Functional Neuroimages).

There are a number of parameters that can be adjusted for each of these packages. In an embodiment, the subject smart phantom can be used to adjust the algorithm, by adjustment of parameters, to achieve an appropriate P value (a threshold that determines which voxel is activated) of the statistical map. In an embodiment where the subject phantom provides motion of the sample material, RF coils, DC coils, and/or the entire phantom, the best motion correction parameters, such as basis choice and interpolation methods can be arrived at. In an embodiment where the phantom provides physiological noise, such as respiratory and cardiac perturbation, the best parameters of temporal smoothing can be arrived at.

Design Scheme I

In a specific embodiment, the subject phantom can provide four or more RF probes/coils. In a further specific embodiment, each loop can be about 2.5 cm×5 cm. In alternative embodiments, and RF coil has a maximum length in at least one dimension of less than 4 cm. and/or a maximum length in at least one dimension of less than one-third the maximum length of the sample in the same dimension. Each RF loop can be strategically placed in the phantom such that the magnetic field associated with driving a current through the RF loop has at least a component perpendicular to the static magnetic field B₀ of the MR scanner into which the subject phantom is placed. In an embodiment, the loop(s) can be placed parallel with the static magnetic field Bₒ. In an embodiment utilizing a head phantom, coils can be placed at positions that approximate the location of the primary visual cortex, the location of the motor cortex, and the location of the auditory cortex. The probes act as local spin magnetization amplifiers and thus produce regions of variable MR image contrast to noise.

The control signal can be produced by a personal computer (PC), and can provide, for example, the following functions:

(1) A range of enhancement level from, for example, 1-10% can be modeled over each region of interest. In a specific embodiment, enhancement levels of 0.25% -1% can be modeled as well. Positive and/or negative enhancement levels can be produced, as can enhancement levels higher than 10%.

(2) Typical BOLD modeling (Poisson, Gamma, Gaussian)

The fMRI time series signal (hemodynamic response) at time t due to neuronal activity x(t) (sensory or cognitive stimulation) is denoted by y(t), the coupling between the neuronal activity and hemodynamic response is given by
y(t)=γx(t)·h(t)+n(t) (1)
where ‘·’ denotes convolution, γ denotes the gain of the fMRI imaging process and n(t) represents the noise. The h(t) is the hemodynamic modulating function or hemodynamic “impulse response” of the BOLD signal. The h(t) has been modeled as different functions, such as a Poisson function, a Gamma function, and a Gaussian function. Therefore, these, and/or other, functions can be employed in the BOLD signal modeling for the phantom. The mathematical forms of these functions can be represented as follows:
a. Poisson function

$$h ( t ) = \frac{{λ}^{t} {ⅇ}^{- λ}}{t !} \qquad ( 2 )$$

where λ represents the lag and dispersion.
b. Gaussian function

$$h ( t ) = \frac{1}{\sqrt{2 {πσ}^{2}}} exp ( - \frac{{( t - μ )}^{2}}{{σ}^{2}} ) \qquad ( 3 )$$

where μ and σ are the mean and standard deviation (or lag and dispersion) of the function.
c. Gamma function

$$h ( t ) = \frac{{( t / τ )}^{n - 1} {ⅇ}^{- t / τ}}{τ ( n - 1 ) !} \qquad ( 4 )$$

where t is time, τ is a time constant, and n is a phase delay. A pure delay between stimulus onset and the beginning of the fMRI response is also allowed. The pure delay here accounts for any systematic asynchrony between stimulus onset and data acquisition and for any real delay between stimulus onset and hemo-dynamic response.

Most fMRI studies have been focused on neural activation analysis and have showed the timing issue. As reviewed in Chapter 19 of Moonen & Bandettini (Eds.) (1999) Functional MRI, Springer-Verlag, with activation, the time for the BOLD response to first significantly increase from baseline is approximately 2 s. The time to plateau in the on state is approximately 6-9 s. With cessation of activation, the time to return to baseline is longer than the rise time by about 1 or 2 s. Several groups have reported a pre-undershoot or initial dip during the first 500 ms to 2 s of the signal. More commonly observed is a post-undershoot, which is observed more in visual cortex than in motor cortex and has an amplitude that is dependent on stimulus duration.

The control waveform of the subject smart phantom can be modeled to reflect the dynamic characteristics according to the research in the literature. Duty cycle control can also be done through the waveforms sent to the phantom. For example, the on-off duration can be from 50%-50% to 10%-90%.

In a specific embodiment of the subject phantom, physiological “noise”, i.e., cardiac, respiratory, and brain (for example around 0.1 Hz) “noise” can be modeled and simulated.

Validation of the Subject Smart Phantom

The desired “answer” in blood oxygenation level dependent (BOLD) fMRI is delineation of an enhancement region with the best confidence. The subject phantom can serve as a local signal enhancement model.

The following process can be used in accordance with the subject invention. A reciprocal view of the coil and phantom can be taken, so that a given coil can be assumed to be driven. The field produced by the MR coil can be assumed constant over the secondary coils (in the phantom) surface area. An EMF can be calculated in the secondary coil. As an example, a simple loop can be used. This induced voltage is 90 degrees out of phase with the source field (coil current). The impedance of the secondary coil can be adjustable to limit the current. In a specific embodiment, the secondary coil impedance can be primarily real, such that the induced current will be 90 degrees out of phase with the source current. Therefore, the net field is the vector sum of B_{M} and B₂, where B_{M}=μₒM, where M is the rotating component of the magnetization of the sample at detection and μₒ is the magnetic permeability of free space, and B₂ is the induced field. It should be noted that although the analysis described is performed using a reciprocal approach, the secondary loop, or RF coil, is preferably deactivated during transmit such that the enhancement of field described applies to the receive phase only. This enables a linear approach of field and sensitivity. Preferably, the RF coils impedence is increased to a high enough value that negligible current flows during the transmit of the excitation magnetic field B₁, where the excitation field B₁ is turned on and off. When the excitation field is off, the impedence of the RF coil(s) can be lowered such that current can flow and stimulated data can be measured. The impedence of the RF coil(s) can be adjusted in a time dependent manner, in order to, for example, model physiological processes.

Circuit Schematic and Impedance Control

Circuit schematics with respect to a specific embodiment of the subject invention are shown in FIGS. 4A and 4B. Each circuit has a conductor loop, and other components that are used to adjust the loop's impedance. The loop is tuned to the Larmor frequency of the magnet with a tuning capacitor, such as C₀ or C₁. The area covered by the loop determines the Region of Interest (ROI), where signal enhancement can be detected by a receive coil, such as a head coil. The signal enhancement is caused by current induced from the sample magnetization during the receive phase.

MRI signals come from the magnetization of nuclei spins of the sample material. The rotation of the magnetization, after excitation of the sample material, induces current in the RF coil. This current in the RF coil produces an induced magnetic field B₂ in the region of interest. This time-varying magnetic field B₂ penetrates the receive, or detection, coil, and generates additional signal in the detector (receive coil) in superposition to the signal from spin magnetization. In the ‘off’ state, where the RF coil is decoupled, only B_{M} induce voltages in the receive coil, resulting in V_{M}. In the ‘on’ state, Bₙₑₜ, instead of B_{M}, will induce voltages in the receive coil, resulting in Vₙₑₜ. Then we can define the enhancement level as:

$$enhancement = \frac{{V}_{net} - {V}_{M}}{{V}_{M}} . \qquad ( 5 )$$

By adjusting the impedance of the loop, which will be pure resistance when resonant, one can change the induced current, which is a ratio of the induced voltage over impedance. The induced current determines the secondary, or induced, field B₂ and, therefore, the level of enhancement, where the enhancement level is defined as

$$\frac{{V}_{net} - {V}_{M}}{{V}_{M}} .$$

FIG. 4A shows an embodiment of a coil and accompanying circuitry implementing impedance control using two LC resonance traps, in accordance with the subject invention. Each trap includes an inductor, a capacitor, and a pin diode. The pin diode is a non-linear component, such that its forward resistance is a function of bias current. The upper trap, including inductor L₁ and capacitor C₁, which are tuned to resonate at the Larmor frequency of the magnet, and diode D₁, produces high impedance during transmit so as to de-couple the resonance loop during transmit. The lower trap, including the inductor L₂ and capacitor C₂, which are tuned to resonate at the Larmor frequency of the magnet, and diode D₂, produces adjustable impedance during receive. By adjusting the bias current fed to diode D₂ through an adjustable resistor R, one can change forward resistance of diode D₂, denoted as r. Therefore, the impedance Zᵣ across capacitor C₂ is

$${Z}_{r} = \frac{{X}_{c}^{2}}{r} \qquad ( 6 )$$

where X_{c} is the reactance of capacitor C₂. Consequently the induced current as well as the enhancement is changed. Inductors L₃, L₄ can function as RF chokes.

FIG. 4B shows another embodiment of a coil and accompanying circuitry implementing impedance control using a resistor array, composed of resistors R₁, R₂and R₃, in accordance with the subject invention. Each resistor is in parallel with a corresponding diode and a corresponding dc-blocking capacitor such that resistor R₁ is in parallel with diode D₂ and capacitor C₂, resistor R₂ is in parallel with diode D₃ and capacitor C₃, and resistor R₃ is in parallel with diode D₄ and capacitor C₄. Therefore, each resistor can be involved into the resonant loop if the corresponding diode is off, and not involved in the resonant loop if the corresponding diode is on. Diode D₁ is reverse-biased during transmit to de-couple the resonance loop. Inductors L₁ through L₄ can function as RF chokes.

Control of Loop Impedance

This section describes how to control the impedance of the resonance loop in order to acquire different levels of enhancement. Referring to FIG. 4A, the impedance of the resonance loop can be controlled by using, for example, a resonance trap or a resistor array. Both of these techniques can incorporate pin diodes that can be biased to control the impedance of the loop. Therefore, by adjusting the biasing current, one can control the impedance of the loop. In the following, two specific implementation methods in accordance with the subject invention, that determine how to send a biasing current so as to control the impedance of the loop, are shown.

1. Control Implementation of Resistor Array

In a specific embodiment, a personal computer (PC)'s serial port/RS232 can be used to send a digital data stream to the subject phantom, as shown in FIG. 5. The data stream includes 1's and 0's, where, for example, a 1 can turn on a diode in the circuitry of the embodiment shown in FIG. 4B so as to bypass the parallel resistor in the loop and a 0 can turn off a diode in the circuitry of the embodiment shown in FIG. 4B such that the parallel resistor is part of the loop impedance.

2. Control Implementation of High Impedance Trap

In another specific embodiment, a PC's parallel port, for example, a sound card, can be used to send a continuous analog signal to the subject phantom. The analog signal can provide a continuous current that biases the diode shown in the embodiment shown in FIG. 4A, which, consequently, controls the impedance of the loop.

Susceptibility Issues in Smart Phantom

Inhomogeneous B-field and EPI Consideration

The subject phantom can be utilized with fMRI informatics. EPI imaging is used intensively in fMRI. EPI images are very sensitive to susceptibility, which can be interpreted as two effects. The first effect is in-plane displacement and distortion caused by off-resonance in the image plane. The second effect is signal dropout due to field in-homogeneity perpendicular to the image plane. In an embodiment, the subject phantom can be designed as a cylinder with gel inside. The field distortion can occur in both transverse direction and longitudinal direction. The size of the phantom can be determined to optimize the EPI image.

In the following, the theoretical issue will be addressed first. If we write
H=−∇Ψ, (7)
where H is magnetic field intensity and Ψ is scalar magnetic potential.

Then,

$${∇}^{2} Ψ = - \frac{{ρ}_{m}}{{μ}_{0}} , \qquad ( 8 )$$

where ρₘ is magnetic charge density and μ₀ is permeability of free space, where

$$\begin{matrix}{ρ}_{m} = - ∇ \cdot ( {μ}_{0} \vec{M} ) \\ = - ∇ \cdot ( \frac{χ}{1 + χ} B ) \\ = - \frac{{μ}_{0}^{2}}{μ} \vec{H} \cdot ∇ χ ,\end{matrix} \qquad ( 9 )$$

where M̅ is magnetization, χ is susceptibility, μ is permeability of the material, and B is magnetic field flux density.

For a cylinder in another media, χ is constant except at the border, so we obtain at point (r, θ, z), in cylindrical coordinates,

$$Ψ = \frac{{μ}_{0}}{4 π μ} {\int }_{S} \frac{{H}_{0} Δχ \cdot ρ}{\sqrt{{r}^{2} + {ρ}^{2} - 2 r ρ cos ( θ - ϕ ) + {z}^{2}}} ⅆ ρ ⅆ ϕ , \qquad ( 10 )$$

where S represents the surface of the top and bottom, ρ is an integral variable for radius, φ is an integral variable for angle, and H₀ is the external magnetic field intensity.

The z component of H is

$$\begin{matrix}{H}_{z} = - \frac{\partial Ψ}{\partial z} \\ = \frac{{μ}_{0}}{4 πμ} {\int }_{S} \frac{z {H}_{0} Δχ \cdot ρ}{{( \sqrt{{r}^{2} + {ρ}^{2} - 2 r ρcos ( θ - ϕ ) + {z}^{2}} )}^{3}} ⅆ ρ ⅆ ϕ\end{matrix} \qquad ( 11 )$$

In this equation θ can be set to zero due to symmetry. Then,

$$\begin{matrix}{H}_{z} = - \frac{\partial Ψ}{\partial z} \\ = \frac{{μ}_{0}}{4 πμ} {\int }_{S} \frac{z {H}_{0} Δχ \cdot ρ}{{( \sqrt{{r}^{2} + {ρ}^{2} - 2 r ρcosϕ + {z}^{2}} )}^{3}} ⅆ ρ ⅆ ϕ\end{matrix} \qquad ( 12 )$$

Simulation

A simulation has been performed for a cylindrical phantom of fixed length, L=20 cm, in z direction. The radius was set to 6 cm, 8 cm, and 10 cm. These radii are consistent with practical situations. FIGS. 6A and 6B show the field distortions in the radial and longitudinal directions, respectively, for radii of 6 cm, 8 cm, and 10 cm. Referring to FIGS. 6A and 6B, for a fixed length cylinder, longitudinal inhomogeneity decreases with radius while transverse inhomogeneity increases with radius. Therefore, there is essentially a tradeoff between the two susceptibility effects. Moving the curves to the same starting point, within the vicinity of z=0 and r=0, the field inhomogeneity is essentially the same. The biggest change happens near the edge of phantom. Since the slices are chosen to be close to the center, it may be preferable to use a 6 cm radius phantom. FIGS. 6A and 6B show field distortions in the radial and longitudinal directions, respectively, for a simulation of a cylindrical phantom of fixed length in the z direction.

FIGS. 7A and 7B show a comparison of the field distortions for three radius by moving the curves in FIGS. 6A and 6B, respectively, to coincide at r=0 or z=0.

With this design, high quality EPI images can be obtained.

FIGS. 8A and 8B show an EPI (SLT=5 mm, TE=54, FOV=160 mm) image and a Spin-echo image (same slice, TR/TE=552/16) of a specific embodiment of the subject smart phantom acquired in a Siemens Symphony system.

Gel Preparation

In an embodiment, the subject phantom can be filled partially, or completely, with a gel. Preferably, the gel in the phantom is chosen to model tissue to be imaged in the imaging system.

Some gels have bubbles, which can cause severe distortions of the EPI image. In a specific embodiment, agarose gel can be used, due to the uniformity and stability of the agarose gel. The T1 and T2 of agarose gel can be controlled by adjusting the concentration of agarose and copper sulfate, where T1 is longitudinal relaxation time and T2 is transverse relaxation time. Measurements of the T1 and T2 of agarose gel under different concentrations of agarose and copper sulfate, respectively, were made. These measurements show that the inverse of T1 and the inverse of T2 are proportional to the concentration of agarose (in pecentage) and the concentration of copper sulfate (in mM). In an embodiment of the subject invention, the following equations can be used to model such relationships

$$\begin{matrix}\frac{1}{{T}_{2}} = 9.0 \times {10}^{- 3} [ agarose \% ] + 2.10 \times {10}^{- 3} [ Cu S {O}_{4} ] + 0.97 \times {10}^{- 3} & ( 13 ) \\ \frac{1}{{T}_{1}} = 1.51 \times {10}^{- 4} [ agarose \% ] + 9.433 \times {10}^{- 4} [ Cu S {O}_{4} ] + 8.7 \times {10}^{- 5} & ( 14 )\end{matrix}$$

To obtain a gel with a T1/T2 ratio in the range of a brain, for example, [CuSO₄]=0.75 mM and [Agarose]=1.2 % can be chosen.
Simulation of Respiratory Artifacts

The effect of respiration on fMRI imaging is usually considered as susceptibility changes due to breathing. According to Schenck (Schenck, J. The role of magnetic susceptibility in magnetic resonance imaging: MRI magnetic compatibility of the first and second kinds. Med. Phys. 23 815-50, 1996), a spherical cavity induces a magnetic dipole field which is given by

$$Δ B ( x , y , z ) = \frac{\frac{1}{3} Δχ {B}_{0} {R}^{3} ( 2 {z}^{2} - {x}^{2} - {y}^{2} )}{{( {x}^{2} + {y}^{2} + {z}^{2} )}^{5 \text{/} 2}} \qquad ( 15 )$$

where R is the radius of the cavity, χ is susceptibility, and B₀ is the external magnetic field. This additional field change can be generated by a small loop perpendicular to the magnetic field placed at the same position.

$${B}_{z} ( x , y , z ) = \frac{I π {a}^{2}}{c} \frac{( 2 {z}^{2} - {x}^{2} - {y}^{2} )}{{( {x}^{2} + {y}^{2} + {z}^{2} )}^{5 \text{/} 2}} for z >> a , \qquad ( 16 )$$

where a is the radius of the loop, I is the current flowing in the small loop, and c is the speed of light. This field is very small compared to B₀, therefore, it can be assume that the transverse field will not affect the spins at all. However, we do not want to place the loop far away from the phantom, such that the field distortion becomes slightly different. The above equation can be rewritten as

$${B}_{z} ( x , y , z ) = \frac{I π {a}^{2}}{c} \frac{( 2 {z}^{2} - {r}^{2} )}{{( {r}^{2} + {z}^{2} )}^{5 \text{/} 2}} \qquad ( 17 )$$

where r is the distance between the imaged pixel and the center of the imaging slice. It is known that the susceptibility has two in-plane effects. One effect is shift of the whole image and another effect is distortion of the image. The shift of the whole image is due to the field difference to Bₒ, the distortion is due to the field difference within the subject. In an embodiment of the subject phantom, z is about 10 cm, while for a human body, z is about 35 cm. The induced field change is computed from the above equation (18) for r up to 5 cm. The results are shown in FIGS. 9A and 9B.

FIGS. 9A and 9B show the field distortion for z=10 cm and z=35 cm, normalized at r=0 and the field distortion for z=10 cm and z=35 cm, scaled to make the overall change from r=0 to r=5 close to each other, respectively.

Referring to FIGS. 9A and 9B, both shift and distortion of the image cannot be simulated simultaneously. However, the shift of the image can be corrected by motion correction, such that this effect is not critical. In an embodiment, the respiratory effect can be simulated reasonably well by regulating the current in the additional loop of the subject phantom in a rhythm consistent with breathing.

In an embodiment, the gradient field can induce emf in this loop, and

$$emf = \frac{ⅆ Φ}{ⅆ t} \sim \frac{Δ B}{Δ t} {S}_{loop} . \qquad ( 18 )$$

However, for an EPI image, the effect of the gradient field can be neglected because the z-gradient only happens once for each slice and it typically only affects the first few lines of k-space.

An embodiment of the subject smart phantom has been used to simulate a Blood Oxygenation Level Dependent (BOLD) signal in a real scan. In this scan, 205 single-slice gradient echo EPI images were acquired in a GE 1.5 T Signa scanner with a 4-channel phased array head coil (MRI Devices Corporation, Waukesha, Wis., USA). The field of view (FOV)=16 cm, the Repetition Time (TR)=3 s and the Echo Time (TE)=31 ms. The image Matrix=64×64, and the Slice Thickness (SLT)=3 mm. The first 5 images were discarded. The remaining images follow the paradigm of alternative off-on states, each of which lasts 30 s. The enhancement level of the phantom was set to 2%. The exams were repeated 3 times to get different signal-to-noise (SNR) ratios with Flip Angle (FA) equal to 90°, 30°, and 120. FIG. 10A is an EPI image of an axial slice of the subject phantom. The spin-like signal enhancement is supposed to be localized to the center of the circle and uniform in this region. Therefore, all the analyses are chosen to be constrained on the 9-pixel square region of interest (ROI). FIG. 10B shows the BOLD-similar time course of the mean signal in the ROI for two enhancement levels. Besides signal variation from one time point to another, slight signal drift is also observed in the 10 minutes scan.

Design Scheme II

In a specific embodiment, a coil loop can be positioned perpendicular to the magnetic field B₀, such that DC current inputted into the loop will produce field distortion to B₀, where the distortion decays with distance from the loop. In other embodiments, one or more coils associated with a magnetic field having a component parallel with static field B. can be utilized. The coil(s) can be positioned above, below, and/or within the sample material. The incorporation of coil(s) providing fields in the Bₒ direction can be in conjunction with the use of RF coils as described associated with magnetic fields perpendicular to Bₒ, or can be used without such RF coils. The field distortion can affect the image in two ways. The field distortion can cause a global shift of the image due to B₀ offset. The field distortion can also cause signal loss due to the gradient of field distortion in B₀ direction, which can be used for simulating BOLD effect. The global shift can be compensated for both active and basal states in BOLD imaging by, for example, using a phantom of cylinder shape with a loop attached to each end, where the axis of the cylinder intersects with the centers of the two loops. FIG. 11 shows an embodiment of the subject phantom having a cylindrical shape and having a loop attached to each end of the phantom such that the axis of the cylinder intersects the centers of the two loops. In a specific embodiment, a plurality of DC coil loops can be positioned and provided with current to produce a substantially constant B-field over the region of interest.

Referring to FIG. 11, a DC current can be input to one or both of the end loops to induce local field distortion, which simulates susceptibility effect as BOLD. Diodes can be used to control on and off states. In an embodiment, the ‘on’ state can be such that both loops have currents and the ‘off’ state can be such that only one loop has current, but of 2 times magnitude. The field of the ‘off’ state in this embodiment is more inhomogeneous in z direction than the field of the ‘on’ state, but the offsets of the z component of the magnetic field are almost the same. The embodiment shown in FIG. 11 can be operated in a mode that is T2-dependent. In theory, the coupling between the end loops and receiver coils is small and, therefore, it should not introduce much noise, which is verified by experiments.

Referring to the embodiment shown in FIG. 11, two solenoids (10-turn loops, 2 cm in diameter) were put on the ends of a phantom in accordance with the subject invention, with the axes of the end loops along Bₒ direction. For the ‘on’ state, 300 mA current was driven to both solenoids, and 60 images were acquired. For the ‘off’ state, 600 mA current was driven to only one solenoid, and 60 images were acquired. FIG. 12 shows a difference image from the ‘on’ state (with current in both loops) to the ‘off’ (with two times current in only one loop), after averaging. In FIG. 12, the center of mass shifts upward due to field superposition. If we change the direction of the current, this effect can be, at least, partially corrected.

Other embodiments utilizing one or more coils as shown in FIG. 11 can drive the coils with a time varying current, in order to model time-varying activities. In a specific embodiment, the coil(s) can be driven by a time-varying current that models breathing.

In an embodiment, two small loops can be placed inside an embodiment of the subject phantom. Each of the two loops can be associated with magnetic fields having a component parallel to the static field B₀ in the region of interest. In an embodiment, the two loops are planar, parallel, perpendicular to the magnetic field B₀ (in z direction), and associated with magnetic fields parallel with the B₀ field. DC current can be input to the loops to induce a local field distortion in the Bₒ field. The local field distortion can be used to simulate a susceptibility effect. Susceptibility effects can be used for imaging purposes, such as used in BOLD. An embodiment can make the field of one state more inhomogeneous in the z direction than another state and, therefore, the dephasing effect is different, resulting in a signal difference for the two states. The two loops can be driven with co-rotating currents, which can be referred as a Helmholtz pair, and/or the two loops can be driven with counter-rotating currents, which can be referred as a Maxwell pair.

In an embodiment, with the two loops driven as a Maxwell pair the current flows gives rise to little, or no, distortion of the magnetic field at the midway point between the two loops and a fairly uniform field gradient in the z direction parallel to the Bₒ field in a wide range around the midpoint. The uniform gradient of the distortion in the Bₒ field can facilitate multi-slice MRI acquisition and make the signal change insensitive to slice position.

An embodiment of a phantom can incorporate a sample material having a cylindrical shape. The susceptibility effect due to the sample material in a phantom is added to the Bₒ distortion induced by the DC currents in the, for example, two loops, and gives rise to a complex pattern of signal change depending on the current direction and slice position. In an embodiment, the shape of the sample material can be controlled to control B₀ distortion within the phantom. In an embodiment, the shape of the sample material is selected to achieve a constant B₀ distortion across the ROI. In a specific embodiment, a spherical shaped sample material can be incorporated with the phantom. In theory, the spherical shaped sample material induces a constant B₀ distortion within the phantom, such that there is no additional field gradient from the sample material in the ROI.

FIG. 15 shows a comparison of the magnetic field along the axis of the loops for loops driven as a Maxwell pair and loops driven as a Helmholtz pair. In an embodiment, a coaxial cable can be used to connect the two loops so that the DC paths produce negligible magnetic field due to the currents canceling each other. RF chokes can be incorporated with the loop pair to minimize induced RF current. For this two loop coil structure driven as a Maxwell coil the image intensity is related to the echo time (TE), similar to BOLD imaging. In an embodiment, there is no need to decouple the loops during transmit. The field change for this design is global such that the effect can be sensitive to slice thickness and can be sensitive to slice position.

In an embodiment, steps can be taken to address the shift of the image due to the non-zero B₀ distortion. If the slice position is off the center, or midpoint between the two loops, the distortion of the magnetic field is not zero. An estimate can be made as to how big the shift is due to non-zero B₀ distortion. If the dephasing effect in the B₀ direction is taken into consideration
S∝M sin c(γG_{z}W·TE/2)
where G_{z} is the local field gradient and W is the slice thickness. This can be arrived at from

$$S ∝ {\int }_{- W / 2}^{W / 2} {ⅇ}^{- ⅈ γ TE {G}_{z} z} ⅆ z .$$

In order to produce a 4% signal difference,
γG_{z}·W·TE/2=0.157
so with W=4 mm, TE=40 ms for 1.5 T, and γ=42.577 MHz/T, we obtain G_{z}=46 μT/m. Then 5 mm away from the center, the B₀ distortion is G_{z}*0.005 m=0.23 μT, corresponding to 9.8 Hz bandwidth. Assuming the bandwidth per pixel is 20, the pixel shift caused by this should be around 0.5 pixel. This pixel shift is not desirable, but will not cause much of a signal change in the ROI if the phantom is fairly uniform.

A multi-slice acquisition was conducted with an embodiment of the subject phantom incorporating a spherical shaped sample material and two loops. The two loops of the phantom are 10 cm apart and positioned at the top and bottom of the spherical sample material with the axis of the spherical sample material coaxial with the two loops. During the acquisition 9 slices are acquired with a 4 mm slice thickness and a 1 mm gap. The center of intensity was calculated in the phase encoding direction and the results are shown in FIG. 16. The center of intensity is defined as the ratio of the sum of pixel indices multiplied by the pixel intensity to the mean signal intensity of the image. When we calculate the center of intensity, those pixels with very low signal intensity are excluded because they are mostly noise. The overall pixel shift for the center slice is ˜0.04 pixels, while the most outer slice has a shift of about 0.5 pixels. For the same phantom, the signal change is also calculated as a ratio to the undistorted image for the center slice. The results are displayed in FIGS. 17A-17C. FIG. 17A shows the image of the middle slice without any current driven in the two loops; FIG. 17B shows the image of the same slice with current to drive the two loops as a Maxwell loop; and FIG. 17C shows the difference image, based on the difference between the image of FIG. 17B and the image of FIG. 17A, divided by the image of 17A. It is clear that the signal difference has its maximum at the center, or midpoint between the two loops, and decays rapidly towards the edge. Accordingly, the ROI will not be too big in comparison with brain activation areas.

Another effect is a change of enhancement level with slice position due to in-plane B₀ inhomogeneity. This effect is also very small because the gradient in the transverse plan is less than 1/10 of the gradient in the longitudinal direction. However, both the effect from Bₒ distortion and the change of enhancement level with slice position due to in-plane Bₒ inhomogeneity get bigger as the slice position is farther from the center, or midpoint between the two coils.

A method in accordance with the subject invention to reduce the impact of the effect from B₀ distortion and the change of enhancement level involves acquiring two images with opposite currents, where opposite currents means the two loops are driven in a certain manner during a first image with the loop currents of each loop reversed during a second image. Opposite currents result in the gradient of the B₀ field distortion being the opposite during the second image with respect to the gradient of the B₀ field distortion during the first image. The average of the first image and the second image has a reduced artifact, as shown by the simulation results in one dimension. FIG. 18A shows the profile of the B₀ distortion. FIG. 18B shows a comparison of different signal changes due to the B₀ distortion in FIG. 18A. The B₀ distortion induces a pixel shift that appears as a declining signal on one end and an increasing signal on the other end, as demonstrated in FIG. 18B. As the current is reversed, so is the gradient and the direction of the signal change. If we take the average, an image very close to the true image can be obtained.

In an embodiment, the slice selection gradient can be ˜5-10 mT/m, which is about 100 times G_{z}. In an embodiment, G_{z} is less than or equal to 1/10 of the slice selection gradient; in a further embodiment, G_{z} is less than or equal to 1/50 of the slice select gradient; and in a further embodiment, G_{z} is less than or equal to 1/100 of the slice selection gradient.

1. Comparison with different coils at 1.5 T.

FIG. 19 shows a comparison of the enhancement in the central ROI for a volume coil and an array coil. A phased array coil and volume coil were in a GE Signa scanner to test an embodiment of the phantom. The slice thickness is 4 mm, with 1 mm gap and TR/TE=2000/40. The results of the central 4 slices are shown in FIG. 19, which shows a comparison of contrasts of the phantom used with two different receive coils. There is about 10% difference between the results from different coils.

The subject invention also relates to a method and apparatus for fMRI imaging. In an embodiment, a patient to be imaged can be positioned in an MR scanner, in a region of interest in the static magnetic B₀, and one or more dc coils associated with corresponding magnetic fields having components parallel to the static magnetic field B₀ in the region of interest in the MR scanner. The dc coils can be driven with corresponding time dependent dc currents while imaging the patient. In an embodiment, stimulation can be withheld from the patient during imaging. In alternative embodiments, stimulation can be provided to the patient during the imaging. The fMRI data obtained during the imaging can be used to create an fMRI reference signal with respect to the patient. The patient can then be imaged while driving the dc coils with a second corresponding time dependent dc current while imaging the patient. In a specific embodiment, the second corresponding time dependent dc current is zero for all of the dc coils during imaging of the patient. The patient can be stimulated with a time dependent stimulation, as typically used in fMRI imaging, while the dc current to the dc coils is zero, and imaged to produce an fMRI signal for the patient with respect to the stimulation. The fMRI reference signal can then be used to remove patient dependent artifacts from the fMRI signal. The removal of the artifacts can be accomplished via, for example, subtraction, deconvolution, and/or division.

In a specific embodiment for fMRI imaging a patient's head, a dc coil can be positioned above the patient3 s head where the static magnetic field Bo of the MR scanner is parallel with the patient's neck. Alternatively, or in addition to the dc coil above the patient's head, one or more additional coils can be positioned, for example, around the patient's neck and/or around the patient's head. In other embodiments, the patient's head can be positioned in a different orientation and/or other portions of the patient's body can be imaged.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for producing MRI data, comprising:
locating a sample material in a region of interest in a static magnetic field Bₒ;
locating at least one coil, wherein the at least one coil is associated with a corresponding at least one magnetic field that alters the magnetic field parallel to the static magnetic field Bₒ in the region of interest;
driving the at least one coil with a corresponding at least one time dependent current, wherein the time dependent current has at least two different values; exciting the sample material with an excitation RF magnetic field B₁, which has a component perpendicular to the static magnetic field Bₒ, such that the magnetization of the sample material rotates;
detecting a net RF magnetic field Bₙₑₜ in the region of interest after exciting the sample material with the excitation RF magnetic field B₁, wherein Bₙₑₜ in the region of interest after exciting the sample material is the magnetic field B_{M} of the rotating magnetization M, where B_{M}=μₒM and μₒ is the magnetic permeability of free space, wherein an MRI signal is produced for the region of interest.

2. The method according to claim 1,
wherein at least a portion of the corresponding at least one time dependent current has a value of zero.

3. The method according to claim 1,
wherein the corresponding at least one time dependent current is dynamically varying.

4. The method according to claim 1,
wherein the at least one coil comprises a first planar coil having a normal parallel to Bₒ.

5. The method according to claim 4,
wherein the at least one coil comprises a second planar coil having a normal parallel to Bₒ, wherein the first planar coil and the second planar coil are coaxial.

6. The method according to claim 3,
wherein one or more of the corresponding at least one time dependent current that is dynamically varying models a physiological function.

7. The method according to claim 6,
wherein the physiological function is breathing.

8. The method according to claim 1,
wherein the sample material has magnetic properties that match human brain tissue.

9. The method according to claim 1,
wherein the sample material has a T1 value and a T2 value that match human brain tissue.

10. The method according to claim 1,
wherein the sample material comprises a gel.

11. The method according to claim 1, further comprising:
moving at least a portion of the sample material during detecting a net RF magnetic field Bₙₑₜ in the region of interest.

12. The method according to claim 1, further comprising:
flowing at least a portion of the sample material through a conduit during defecting a net RF magnetic field Bₙₑₜ in the region of interest.

13. The method according to claim 1, further comprising:
changing the shape of the sample material during defecting a net RF magnetic field Bₙₑₜ in the region of interest.

14. The method according to claim 1, further comprising:
locating one or more RF shields to shield at least a portion of the sample material from the excitation RF magentic field B₁.

15. The method according to claim 1,
wherein the sample material has a T1 of approximately 1000 ms and a T2 of approximately 90 ms.

16. The method according to claim 1,
wherein the sample material is a human head.

17. The method according to claim 1,
wherein the sample material has a spherical shape.

18. The method according to claim 5,
wherein the sample material has a cylindrical shape and is located between the first planar coil and the second planar coil.

19. The method according to claim 5,
wherein dc current driving the first planar coil is counter-rotating with respect to the dc current driving the second planar coil.

20. The method according to claim 1,
wherein the sample material has magnetic properties that match human bone tissue.

21. The method according to claim 1,
wherein the sample material has magnetic properties that match human heart tissue.

22. The method according to claim 10,
wherein the sample material comprises an agarose gel.

23. The method according to claim 10,
wherein the gel incorporates one or more substances that alter the T2 and/or T1 values of the gel.

24. The method according to claim 22,
wherein the agarose gel incorporates copper sulfate. J

25. The method according to claim 1,
wherein the sample material comprises water doped with ions, oils, and/or organic material.

26. The method according to claim 25,
wherein the sample material comprises water doped with polyvinyl alcohol.

27. The method according to claim 1,
wherein the at least one coil is associated with a corresponding at least one magnetic field such that each of the corresponding at least one dc magnetic field has a component that is parallel to the static magnetic field Bₒ in a region of interest.

28. The method according to claim 1,
producing simulated fMRI data for the region of interest from the MRI signal.

29. The method according to claim 1,
wherein driving the at least one coil with a corresponding at least one time dependent current alters the MRI signal intensity less than an amount that would cause the loss of spatial information.

30. The method according to claim 1,
wherein the at least one coil with a corresponding at least one time dependent current alters the MRI signal intensity less than 5%.

31. An apparatus for producing MRI data, comprising:
a sample material located in a region of interest in a static magnetic field Bₒ;
at least one coil, wherein the at least one coil is associated with a corresponding at least one magnetic field that alters the magnetic field parallel to the static magnetic field Bₒ in the region of interest;
a means for driving the at least one coil with a corresponding at least one time dependent current, wherein the time dependent current has at least two different values;
a means for exciting the sample material with an excitation RF magnetic field B₁, which has a component perpendicular to the static magnetic field Bₒ, such that the magnetization of the sample material rotates;
a means for detecting a net RF magnetic field Bₙₑₜ in the region of interest after exciting the sample material with the excitation RF magnetic field B₁, wherein Bₙₑₜ in the region of interest after exciting the sample material is the magnetic field B_{M} of the rotating magnetization M, where B_{M}=μₒM and μₒ is the magnetic permeability of free space, wherein an MRI signal is produced for the region of interest.

32. The apparatus according to claim 31,
wherein at least a portion of the corresponding at least one time dependent current has a value of zero.

33. The apparatus according to claim 31,
wherein the corresponding at least one time dependent current is dynamically varying.

34. The apparatus according to claim 31,
wherein the at least one coil comprises a first planar coil having a normal parallel to Bₒ.

35. The apparatus according to claim 34,
wherein the at least one coil comprises a second planar coil having a normal parallel to Bₒ, wherein the first planar coil and the second planar coil are coaxial.

36. The apparatus according to claim 33,
wherein one or more of the corresponding at least one time dependent current that is dynamically varying models a physiological function.

37. The apparatus according to claim 36,
wherein the physiological function is breathing.

38. The apparatus according to claim 31,
wherein the sample material has magnetic properties that match human brain tissue.

39. The method according to claim 31,
wherein the sample material has a T1 value and a T2 value that match human brain tissue.

40. The apparatus according to claim 31,
wherein the sample material comprises a gel.

41. The apparatus according to claim 31, further comprising:
a means for moving at least a portion of the sample material during detecting a net RF magnetic field Bₙₑₜ in the region of interest.

42. The apparatus according to claim 31, further comprising:
a means for flowing at least a portion of the sample material through a conduit during defecting a net RF magnetic field Bₙₑₜ in the region of interest.

43. The apparatus according to claim 31, further comprising:
a means for changing the shape of the samplematerial during detecting a net RF magnetic field Bₙₑₜ in the region of interest.

44. The apparatus according to claim 31, further comprising:
one or more RF shields located to shield at least a portion of the sample material from the excitation RF magnetic field B₁.

45. The apparatus according to claim 31,
wherein the sample material has a T1 of approximately 1000 ms and a T2 of approximately 90 ms.

46. The apparatus according to claim 3,
wherein the sample material is a human head.

47. The apparatus according to claim 31,
wherein the sample material has a spherical shape.

48. The apparatus according to claim 35,
wherein the sample material has a cylindrical shape and is located between the first planar coil and the second planar coil.

49. The apparatus according to claim 35,
wherein dc current driving the first planar coil is counter-rotating with respect to the dc current driving the second planar coil.

50. The apparatus according to claim 31,
wherein the sample material has magnetic properties that match human bone tissue.

51. The apparatus according to claim 31,
wherein the sample material has magnetic properties that match human heart tissue.

52. The apparatus according to claim 40,
wherein the sample material comprises an agarose gel.

53. The apparatus according to claim 40,
wherein the gel incorporates one or more substances that alter the T2 and/or T1 values of the gel.

54. The apparatus according to claim 52,
wherein the agarose gel incorporates copper sulfate.

55. The apparatus according to claim 31,
wherein the sample material comprises water doped with ions, oils, and/or organic material.

56. The apparatus according to claim 55,
wherein the sample material comprises water doped with polyvinyl alcohol.

57. The apparatus according to claim 31,
wherein driving the at least one coil with a corresponding at least one time dependent current alters the MRI signal intensity less than an amount that would cause the loss of spatial information.

58. The apparatus according to claim 31,
wherein driving the at least one coil with a corresponding at least one time dependent current alters the MRI signal intensity less than 5%.

59. A method for producing simulated fMRI data, comprising:
locating a sample material in a region of interest in a static magnetic field Bₒ;
locating a coil in a static magnetic field Bₒ, wherein the coil is associated with a corresponding dc magnetic field such that the corresponding dc magnetic field has a component that is parallel to the static magnetic field Bₒ in the region of interest;
driving the coil with a corresponding time dependent current, wherein the corresponding time dependent current has at least two different magnitudes;
exciting the sample material with an excitation RF magnetic field B₁, which has a component perpendicular to the static magnetic field Bₒ, such that the magnetization of the sample material rotates;
detecting a net RF magnetic field Bₙₑₜ in the region of interest after exciting the sample material with the excitation RF magnetic field B₁, wherein Bₙₑₜ in the region of interest after exciting the sample material is the magnetic field B_{M} of the rotating magnetization M, where B_{M}=μₒM and λₒ is the magnetic permeability of free space; and
producing simulated fMRI data for the region of interest.

60. A method for fMRI imaging, comprising:
locating a patient in a region of interest in a static magnetic field Bₒ;
locating at least one coil, wherein the at least one coil is associated with a corresponding at least one magnetic field that alters the magnetic field parallel to the static magnetic field Bₒ in the region of interest;
driving the at least one dc coil with a corresponding at least one time dependent dc current, wherein each of the corresponding at least one time dependent dc current has at least two different values;
exciting the patient to be imaged sample material with an excitation RF magnetic field B₁, which has a component perpendicular to the static magnetic field Bₒ, such that the magnetization of the sample material rotates;
detecting a net RF magnetic field Bₙₑₜ in the region of interest after exciting the patient to be imaged with the excitation RF magnetic field B₁, wherein Bₙₑₜ in the region of interest after exciting the patient to be imaged is the magnetic field B_{M} of the rotating magnetization M, where B_{M}=μₒM and μₒ is the magnetic permeability of free space, where a reference fMRI signal is produced for the region of interest;
stimulating the patient to be imaged with a time dependent stimulation;
exciting the patient to be imaged with the excitation RF magnetic field B₁, which has a component perpendicular to the static magnetic field Bₒ, such that the magnetization of the patient to be imaged rotates;
detecting the net RF magnetic field Bₙₑₜ in the region of interest after exciting the patient to be imaged with the excitation RF magnetic field B₁, wherein Bₙₑₜ in the region of interest after exciting the patient to be imaged is the magnetic field B_{M} of the rotating magnetization M, where B_{M}=μₒM and μₒ is the magnetic permeability of free space, wherein a second portion of an MRI signal is produced for the region of interest;

61. The method according to claim 60,
wherein each of the corresponding at least one magnetic field has a component that is parallel to the static magnetic field Bₒ in the region of interest;

62. The method according to claim 60, further comprising: producing fMRI data for the patient to be imaged in the region of interest.

63. The method according to claim 62, further comprising:
removing patient to be image dependent artifacts from the fMRI data via the reference fMRI signal.
